(19) United States
(12) Patent Application Publication
(10) Pub. No.: US 2021/0403989 A1
(43) Pub. Date: Dec. 30, 2021

(54) BARCODING METHODS AND COMPOSITIONS

(71) Applicant: Bio-Rad Laboratories, Inc., Hercules, CA (US)

(72) Inventors: Ronald LEBOFSKY, Berkeley, CA (US); Man CHENG, Danville, CA (US)

(21) Appl. No.: 17/357,618

(22) Filed: Jun. 24, 2021

Related U.S. Application Data
(60) Provisional application No. 63/044,161, filed on Jun. 25, 2020.

(51) Int. Cl. C12Q 1/6834; C12Q 1/6869

(52) U.S. Cl. CPC C12Q 1/6834; C12Q 1/6869

(57) ABSTRACT

Barcoding composition and methods involving solid supports having sets of different oligonucleotides that can be decoded to the same identification sequence.

### CROSS-REFERENCES TO RELATED APPLICATIONS

The present application claims benefit of priority to U.S. Provisional Patent Application No. 63/044,161, filed Jun. 25, 2020, which is incorporated by reference for all purposes.

### REFERENCE TO SUBMISSION OF A SEQUENCE LISTING AS A TEXT FILE

The Sequence Listing written in file 094868-1250031-117510US_SL.txt created on Aug. 19, 2021, 6,178 bytes, machine format IBM-PC, MS-Windows operating system, is hereby incorporated by reference in its entirety for all purposes.

### BACKGROUND OF THE INVENTION

Next generation sequencing technology can provide enormous amounts of sequence information from a relatively small sample, such as a sample of nucleic acid (e.g., genomic DNA or mRNA) from a single cell. Partitions (e.g., droplets) can be used to generate parallel reactions, for example where cells are in different partitions. DNA sequences in different partitions can be tracked by attaching a different barcode per partition, thereby allowing for nucleic acids from different partitions to later be mixed and tracked back to their origin cell due to the presence of different barcodes. In addition, in some cases, the attachment of unique molecular identifiers (UMIs), such as unique oligonucleotide barcode sequences, to target nucleic acids, and detection of such UMIs during sequencing, can allow estimation of absolute or relative abundance of target nucleic acids in a sample and/or can be used to distinguish between copies of a nucleic acid molecule made during the sequencing method and unique nucleic acid molecules in a sample.

One way to deliver barcode oligonucleotides to partitions is to introduce a solid support (e.g., a bead) into partitions, where each solid support carries a large number of identical oligonucleotides having a unique barcode. Once introduced into a partition, the barcode can be associated with genetic material in the partition, thereby generating a partition-specific barcode. One can form a sufficient dilution of solid supports such that based on a Poisson distribution, a large number of partitions contain only one solid support and thus one partition-specific barcode. However, methods also exist for deconvoluting results where two or more barcodes are introduced into the same partition.

### BRIEF SUMMARY OF THE INVENTION

In some embodiments, a solid support is provided comprising multiple copies of a plurality of at least 10 different oligonucleotide members, wherein all oligonucleotide members encode the same family identification sequence, and wherein the oligonucleotide members comprise one or more sequence block having at least three nucleotide positions and comprising the formula (X)ₙ(Y)ₘ or (Y)ₘ(X)ₙ, wherein X is a degenerate nucleotide, n is 2-50 (e.g., 2-20, 3-20, 4-20, 5-20), Y is constant within the oligonucleotide family, and m is 1-50 (e.g., 1-30, 1-20, 1-10, 1-5), wherein the sum of n and m is at least three, wherein degenerate nucleotides in the at least three nucleotide positions are related between oligonucleotide members by a code such that different oligonucleotide members are decoded to the same oligonucleotide family sequence.

In some embodiments, the solid support has between 2-1000 copies of each different oligonucleotide member.

In some embodiments, n is 2 and m is 1.

In some embodiments, the sequence block has the formula Y[(X)ₙ(Y)ₘ]_{z}, wherein z is 1, 2, 3,4 ,5, 6, 7, 8, 9, or 10. In some embodiments, z is 4, n is 2 and m is 1.

In some embodiments, the family identification sequence of each oligonucleotide member is encoded by one or more (e.g., 1, 2 3, 4, 5, or more) sequence block comprising XₙYₘXₙ. In some embodiments, the family identification sequence of each oligonucleotide member is encoded by one or more (e.g., 1, 2 3, 4, 5, or more) sequence block comprising YₘXₙYₘ. In some embodiments, the family identification sequence of each oligonucleotide member is encoded by one or more (e.g., 1, 2 3, 4, 5, or more) sequence block comprising XₙYₘXₙYₘXₙ. In some embodiments, the family identification sequence of each oligonucleotide member is encoded by one or more (e.g., 1, 2 3, 4, 5, or more) sequence block comprising YₘXₙYₘXₙYₘ. In some embodiments, the family identification sequence of each oligonucleotide member is encoded by one or more (e.g., 1, 2 3, 4, 5, or more) sequence block comprising XₙYₘXₙYₘXₙYₘXₙ.

In some embodiments, n is 2 or 3 or 4 and m is 1 or 2.

In some embodiments, the solid support has between 2-1000 (e.g., 2-50 or 2-500) copies of each different oligonucleotide member.

In some embodiments, the oligonucleotide members do not comprise a unique molecular identification (UMI) sequence separate from the family identification sequence.

In some embodiments, oligonucleotide members are composed of two or more (e.g., 2, 3, 4, 5, 6, or more) sequence blocks.

In some embodiments, oligonucleotide members are composed of sequence blocks that are linked via splint oligonucleotides.

In some embodiments, the oligonucleotide members comprise a 3′ poly T sequence In some embodiments, the oligonucleotide members comprise a sequence complementary to a Tn5 adapter (which is optionally A14).

In some embodiments, oligonucleotide members are linked to the solid support. In some embodiments, the solid support is a bead. In some embodiments, the bead is a dissolvable bead that contains the oligonucleotide members. In some embodiments, the dissolvable bead is a hydrogel bead. In some embodiments, the oligonucleotide members are reversibly (releasably) or irreversibly linked to the bead.

Also provided is a composition comprising a plurality of different solid supports as described above or elsewhere herein, wherein different beads have oligonucleotide members from different oligonucleotide families. In some embodiments, the plurality comprises at least 100, 1000, 10000 or more different solid supports. In some embodiments, oligonucleotide family sequence of different solid supports differ from all other oligonucleotide family sequences by at least two nucleotides in the family identification sequence.

Also provided is a composition comprising a plurality of different solid supports, wherein each solid support comprises multiple copies of a plurality of at least 10 different oligonucleotide members and all oligonucleotide members of a solid support encode the same family identification sequence; and wherein each oligonucleotide member comprises one or more sequence block comprising two or more nucleotides, wherein the two or more nucleotides are degenerate nucleotides and related between oligonucleotide members by a code such that different oligonucleotide members are decoded to the same family identification sequence for the solid support to which the oligonucleotide members are associated; wherein different family identification sequences of different solid supports differ from all other family identification sequences for other solid supports by at least two nucleotides.

In some embodiments, the method further comprises distinguishing sequencing reads for independent fusion polynucleotides by comparing family identification sequences, wherein sequencing reads having the same family identification sequence are considered from the same sample polynucleotide. In some embodiments, different partitions contain different beads and wherein after the linking and before the nucleotide sequencing, contents of the partitions are combined, and wherein sequencing reads from different partitions are identified based on the family identification sequence decoded from the sequencing reads.

In some embodiments, the linking comprises polymerase-based extension of 3′ ends of the oligonucleotide members that hybridize to sample polynucleotides.

In some embodiments, the partitions are droplets in an emulsion In some embodiments, the partitions are wells in a microtiter plate.

### BRIEF DESCRIPTION OF THE DRAWINGS

FIG. 1 depicts the construction process of the bead barcode polynucleotide. A universal oligo sequence is bound to the solid support, such as a bead as depicted. Splint sequences are then used to juxtapose and order the block sequences containing the barcodes to the universal oligo sequence, to each other and to the capture oligo sequence, for example on the 3′ end of the oligo, as shown. The gaps in the top strand are ligated to provide a covalently attached linear polynucleotide. The splints are removed prior to the barcoding reaction, as shown.

FIG. 2 depicts a schematic diagram of a plurality of solid supports (ID-f1,f2,f3 . . . fN) wherein each solid support comprises a plurality of different oligonucleotide members (f1-1,2,3,4,5, . . . N and f2-1,2,3,4,5, . . . N), which comprise unique and specific family identification sequences related by code such that different members are decode to the same oligonucleotide family (f1, f2).

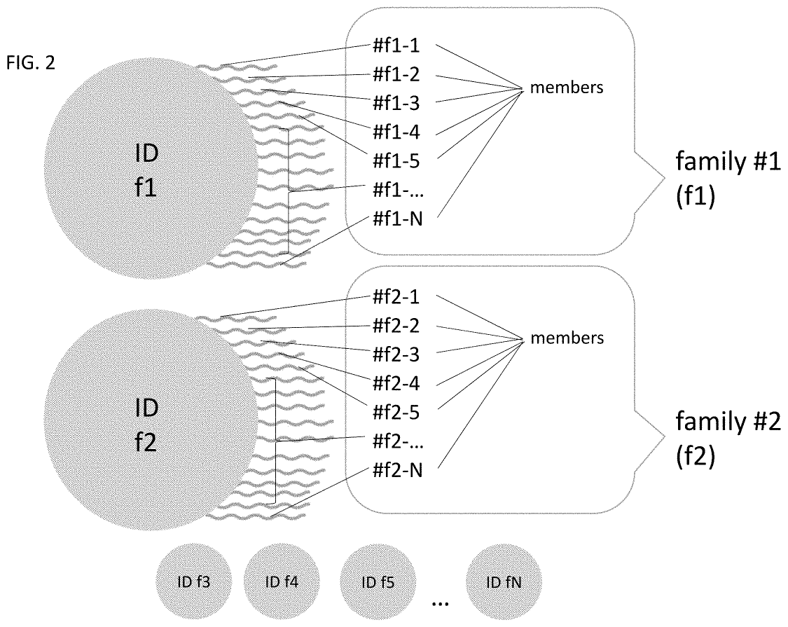

FIG. 3 depicts various scenarios of errors introduced into the described barcoding schemes and how they may be interpreted to read sequences in spite of introduced errors.

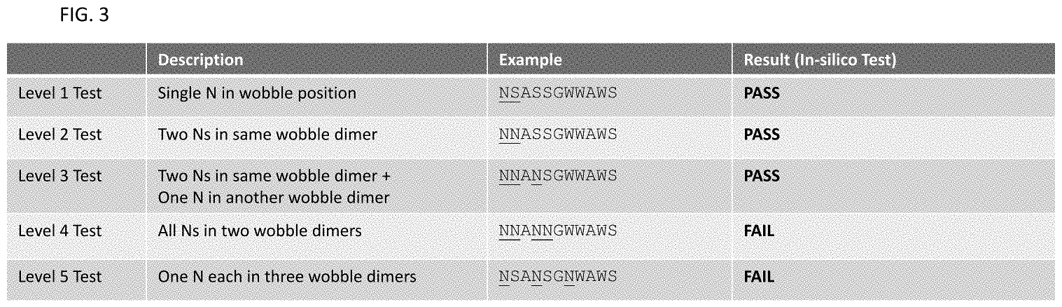

FIG. 4 depicts a combinatorial barcode library construction method. The combinatorial barcode construction method involves tagging, pooling, and splitting steps. Individual barcode-sequence blocks (e.g. 1,2,3,4) are placed in separated well of multi-well plate and subsequently conjugated to solid-support (e.g. Beads). Beads comprising different blocks are then pooled, washed, and redistributed into a new multi-well plate containing the same set of individual barcode-blocks for further conjugation. New barcode sequences (e.g. 1-1,2-1,3-1, . . . 4-4) are created by joining barcode-blocks combinatorially to a mixed pool of barcode-block conjugated beads. The process of tagging, pooling, and splitting steps is repeated multiple rounds until a desired barcode library diversity is achieved. Full-length barcode created by the random combinatorial process is therefore unique and specific to every individual bead of the pool.

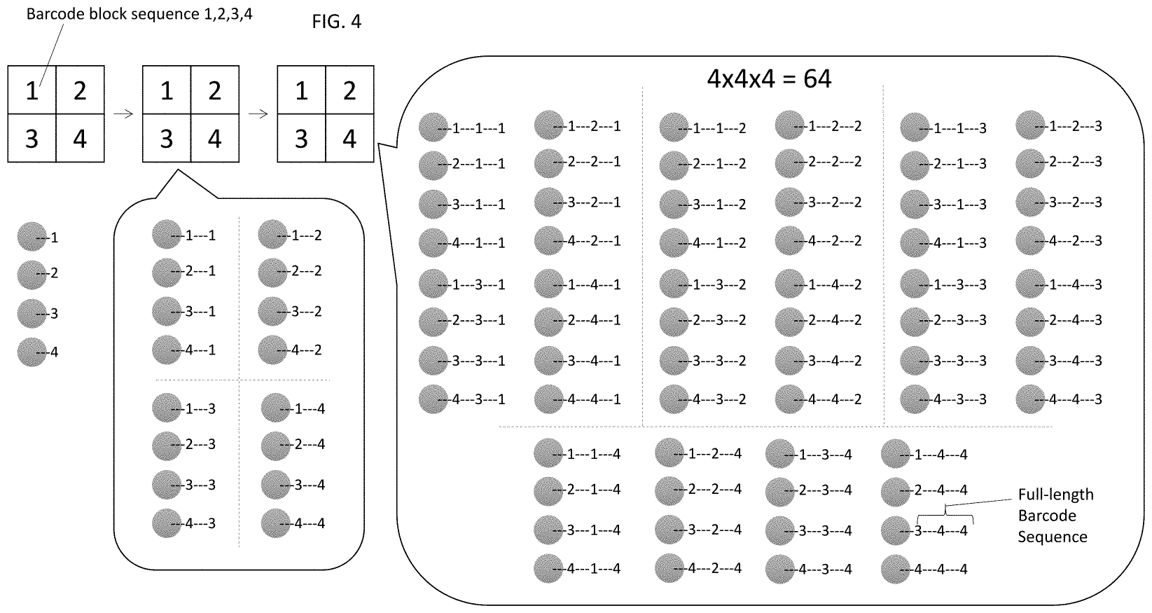

FIG. 5 illustrates a single-cell knee-plot indicating barcode of (X)m(Y)n design scheme be can deconvoluted and directly applied for single-cell identification. The x-axis shows the number of unique barcode in descending order by count of sequencing reads of DNA fragments. The y-axis shows the frequency of reads of DNA fragments associated with a particular barcode. Comparing the frequency of DNA fragments between different barcodes in descending order, a “knee” threshold can be determined as a sharp decrease of the frequency of sequencing reads. The algorithmically-defined threshold indicating a cut-off of a higher number of single-cell DNA fragments reads over a lower number of background DNA fragments reads, thus the knee threshold inferred to represent the single-cell number in the sample.

FIG. 6 depicts knee plots as described in Example 7.

### DEFINITIONS

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization described below are those well-known and commonly employed in the art. Standard techniques are used for nucleic acid and peptide synthesis. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. The nomenclature used herein and the laboratory procedures in analytical chemistry, and organic synthetic described below are those well-known and commonly employed in the art.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a bead” includes a plurality of such beads and reference to “the sequence” includes reference to one or more sequences known to those skilled in the art, and so forth.

“Degenerate” positions or “degenerate nucleotides” are used herein in their common usage and mean that at the position of the nucleotide in question, two or more specific nucleotides (e.g., A, C, G, T) are interpreted based on a code to mean the same thing. In other words, structurally dissimilar nucleotides or nucleotide sequences are interpreted to indicate the same bit of information.

A “constant” nucleotide or nucleotide sequences as used herein refers to a designated nucleotide position, or positions in the case of a constant sequence, in an oligonucleotide as described herein, wherein the same nucleotide occurs at that position in all oligonucleotides attached to a particular solid support. Constant nucleotide positions can be positioned at a known distance (adjacent or otherwise) from one or more variable nucleotides of a barcode so that one can identify where in a sequence read a variable position is. For example, in one example YXXYXXY is a barcode sequence where each Y is a constant nucleotide and X are variable nucleotides. For example, sequence reads might include the following based on the above example: AXXTXXG, where in this case A, T, and G always occur at these positions and nucleotides designated in this example as “XX”) represent the variable degenerate nucleotides making up all or part of the barcode. In some embodiments, the underlying encoded nucleotide is constant while the position in the oligonucleotide is degenerate. For example, in some embodiments, the encoded barcode might be WXXWXXW, where W is can be A or T but in either case the underlying encoded sequence is WXXWXXW.

The term “oligonucleotide family” refers to a set of oligonucleotides associated with a particular solid support and that have the same underlying encoded family barcode sequence that can be distinguished from underlying encoded family barcodes of other solid supports. By “underlying encoded family barcode” is meant the barcode encoded by a degenerate barcode sequence on the oligonucleotide wherein a known code is applied to translate the degenerate barcode to the encoded underling encoded family barcode. The underlying encoded family barcode will be the same for all oligonucleotides associated with particular solid support and will be different for oligonucleotides between solid supports,

The term “solid support” encompasses solid material separated by liquid (such as a bead) or a solid feature (such as a micro-wall separating two wells) that separates liquid in one well from another.

A “family identification sequence” refers to a sequence that indicates the origin to a particular solid support from which the sequence originated. A family identification sequence is a degenerate sequence such that multiple difference sequences can encode the family identification sequence as explained herein. As a basic example, if W=A or T and S=C or G, then WW, SW, WS, and SS can each be different family identification sequences and each can be encoded by multiple sequences. For example, WW can be encoded by AA, AT, TA, or TT and SW can be encoded by GA, GT, CA, and CT. In this basic example there can be four family identification sequences. Where the family identification sequence is longer, more different family identification sequences can be generated.

“Related between members by a code” refers to the code for the degeneracy of the family identification sequence.” In the example above, the code is that W=A or T and S=G or C. By applying the code, the family identification code can be determined and thus oligonucleotides with different sequences can encode the same family identification sequence.

An “oligonucleotide” is a polynucleotide. Generally oligonucleotides will have fewer than 250 nucleotides, in some embodiments, between 4-200, e.g., 10-150 nucleotides.

The term “amplification reaction” refers to any in vitro means for multiplying the copies of a target sequence of nucleic acid in a linear or exponential manner. Such methods include but are not limited to polymerase chain reaction (PCR); DNA ligase chain reaction (see U.S. Pat. Nos. 4,683,195 and 4,683,202; PCR Protocols: A Guide to Methods and Applications (Innis et al., eds, 1990)) (LCR); QBeta RNA replicase and RNA transcription-based amplification reactions (e.g., amplification that involves T7, T3, or SP6 primed RNA polymerization), such as the transcription amplification system (TAS), nucleic acid sequence based amplification (NASBA), and self-sustained sequence replication (3SR); isothermal amplification reactions (e.g., single-primer isothermal amplification (SPIA)); as well as others known to those of skill in the art.

“Amplifying” refers to a step of submitting a solution to conditions sufficient to allow for amplification of a polynucleotide if all of the components of the reaction are intact. Components of an amplification reaction include, e.g., primers, a polynucleotide template, polymerase, nucleotides, and the like. The term “amplifying” typically refers to an “exponential” increase in target nucleic acid. However, “amplifying” as used herein can also refer to linear increases in the numbers of a select target sequence of nucleic acid, such as is obtained with cycle sequencing or linear amplification. In an exemplary embodiment, amplifying refers to PCR amplification using a first and a second amplification primer.

As used herein, “nucleic acid” means DNA, RNA, single-stranded, double-stranded, or more highly aggregated hybridization motifs, and any chemical modifications thereof. Modifications include, but are not limited to, those providing chemical groups that incorporate additional charge, polarizability, hydrogen bonding, electrostatic interaction, points of attachment and functionality to the nucleic acid ligand bases or to the nucleic acid ligand as a whole. Such modifications include, but are not limited to, peptide nucleic acids (PNAs), phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Nucleic acids can also include non-natural bases, such as, for example, nitroindole. Modifications can also include 3′ and 5′ modifications including but not limited to capping with a fluorophore (e.g., quantum dot) or another moiety.

The term “sample nucleic acid” refers to a polynucleotide such as DNA, e.g., single stranded DNA or double stranded DNA, RNA, e.g., mRNA or miRNA, or a DNA-RNA hybrid. DNA includes genomic DNA and complementary DNA (cDNA).

A nucleic acid, or a portion thereof, “hybridizes” to another nucleic acid under conditions such that non-specific hybridization is minimal at a defined temperature in a physiological buffer (e.g., pH 6-9, 25-150 mM chloride salt). In some cases, a nucleic acid, or portion thereof, hybridizes to a conserved sequence shared among a group of target nucleic acids. In some cases, a primer, or portion thereof, can hybridize to a primer binding site if there are at least about 6, 8, 10, 12, 14, 16, or 18 contiguous complementary nucleotides, including “universal” nucleotides that are complementary to more than one nucleotide partner. Alternatively, a primer, or portion thereof, can hybridize to a primer binding site if there are fewer than 1 or 2 complementarity mismatches over at least about 12, 14, 16, or 18 contiguous complementary nucleotides. In some embodiments, the defined temperature at which specific hybridization occurs is room temperature. In some embodiments, the defined temperature at which specific hybridization occurs is higher than room temperature. In some embodiments, the defined temperature at which specific hybridization occurs is at least about 37, 40, 42, 45, 50, 55, 60, 65, 70, 75, or 80° C. In some embodiments, the defined temperature at which specific hybridization occurs is 37, 40, 42, 45, 50, 55, 60, 65, 70, 75, or 80° C. For hybridization to occur, the primer binding site and the portion of the primer that hybridizes will be at least substantially complementary. By “substantially complementary” is meant that the primer binding site has a base sequence containing an at least 6, 8, 10, 15, or 20 (e.g., 4-30, 6-30, 4-50) contiguous base region that is at least 50%, 60%, 70%, 80% , 90%, or 95% complementary to an equal length of a contiguous base region present in a primer sequence. “Complementary” means that a contiguous plurality of nucleotides of two nucleic acid strands are available to have standard Watson-Crick base pairing. For a particular reference sequence, 100% complementary means that each nucleotide of one strand is complementary (standard base pairing) with a nucleotide on a contiguous sequence in a second strand.

As used herein, the term “partitioning” or “partitioned” refers to separating a sample into a plurality of portions, or “partitions.” Partitions are generally physical, such that a sample in one partition does not, or does not substantially, mix with a sample in an adjacent partition. Partitions can be solid or fluid. In some embodiments, a partition is a solid partition, e.g., a microchannel. In some embodiments, a partition is a fluid partition, e.g., a droplet. In some embodiments, a fluid partition (e.g., a droplet) is a mixture of immiscible fluids (e.g., water and oil). In some embodiments, a fluid partition (e.g., a droplet) is an aqueous droplet that is surrounded by an immiscible carrier fluid (e.g., oil).

As used herein a “barcode” is a short nucleotide sequence (e.g., at least about 4, 6, 8, 10, 12, 15, 20, 50 or 75 or 100 nucleotides long or more) that identifies a molecule to which it is conjugated or from the partition in which it originated. Barcodes can be used, e.g., to identify molecules originating in a partition as later sequenced from a bulk reaction. As explained herein, the family identification sequence can be the barcode. Such a partition-specific barcode can be unique for that partition as compared to barcodes present in other partitions. For example, partitions containing target RNA from single-cells can be subject to reverse transcription conditions using primers that contain different partition-specific barcode sequence in each partition, thus incorporating a copy of a unique “cellular barcode” (because different cells are in different partitions and each partition has unique partition-specific barcodes) into the reverse transcribed nucleic acids of each partition. Thus, nucleic acid from each cell can be distinguished from nucleic acid of other cells due to the unique “cellular barcode.” In some cases, substrate barcode is provided by a barcode delivered to the partition on a solid support, e.g., a bead or particle (also referred to as “bead-specific barcode”) or a well, that is present on oligonucleotides associated with the solid support, wherein the family identification sequence is shared by (e.g., identical or substantially identical amongst) all, or substantially all, of the oligonucleotides associated with that particle. As explained herein, in the methods and compositions described herein, the underlying encoded family identification sequence acts as a barcode identical between oligonucleotides associated with the particular solid support though the actual oligonucleotide sequences can be different due to the degenerate nature of the barcodes. Thus solid support-specific barcodes can be present in a partition, attached to a particle, or bound to cellular nucleic acid as multiple copies of the same underlying family barcode sequence.

In some embodiments described herein, barcodes described herein uniquely identify the molecule to which it is conjugated. Because of the degenerate nature of the oligonucleotides described herein on the solid support, a large number of different oligonucleotide sequences are introduced into the same partition. Thus, many if not all copies of a sample nucleic acid will receive a different barcode, allowing for individual marking of separate molecules in the partition. While some sample molecules may be tagged with the identical barcode sequence, the chances of this can be very low and this will not significantly affect the ability to track different copies of a molecule and/or count the molecules. After barcoding, partitions can then be combined, and optionally amplified, while maintaining virtual partitioning (meaning the sequences can be mixed but retain a separate barcode to track their partition origins). Thus, e.g., the presence or absence of a target nucleic acid (e.g., reverse transcribed nucleic acid) comprising each barcode can be counted (e.g. by sequencing) without the necessity of maintaining physical partitions.

The length of the underlying barcode sequence determines how many unique samples can be differentiated. For example, a 1 nucleotide barcode can differentiate 4, or fewer depending on degeneracy, different partitions; a 4 nucleotide barcode can differentiate 4⁴ or 256 partitions or less; a 6 nucleotide barcode can differentiate 4096 different partitions or less; and an 8 nucleotide barcode can index 65,536 different partitions or less.

Barcodes can be synthesized and/or polymerized (e.g., amplified) using processes that are inherently inexact. Thus, barcodes, including underlying family barcodes) that are meant to be uniform (e.g., a cellular, substrate, particle, or partition-specific barcode shared amongst all barcoded nucleic acid of a single partition, cell, or bead) can contain various N−1 deletions or other mutations from the canonical barcode sequence. Thus, barcodes that are intended to be “identical” or “substantially identical” copies can sometimes include barcodes that differ due to one or more errors in, e.g., synthesis, polymerization, or purification errors, and thus contain various N−1 deletions or other mutations from the canonical barcode sequence. Moreover, the random conjugation of barcode nucleotides during synthesis using e.g., a split and pool approach and/or an equal mixture of nucleotide precursor molecules, can lead to low probability events in which a barcode is not absolutely unique (e.g., different from all other barcodes of a population or different from barcodes of a different partition, cell, or bead). However, such minor variations from theoretically ideal barcodes do not interfere with the high-throughput sequencing analysis methods, compositions, and kits described herein. Moreover, as discussed below, underlying family barcodes can be designated such that different barcodes for different solid supports can be designed so that they differ from the closest related underlying family barcode by two or three or more nucleotides thereby allowing for detection in minor (e.g., 1, 2, 3) errors that can arise during sequencing and sample preparation and nevertheless allowing to accurate determination of the origin partition.

In some cases, issues due to the inexact nature of barcode synthesis, polymerization, and/or amplification, are overcome by oversampling of possible barcode sequences as compared to the number of barcode sequences to be distinguished (e.g., at least about 2-, 5-, 10-fold or more possible barcode sequences). For example, 10,000 cells can be analyzed using a cellular barcode having 9 barcode nucleotides, representing 262,144 possible barcode sequences. The use of barcode technology is described in for example Katsuyuki Shiroguchi, et al. Proc Natl Acad Sci USA., 2012 Jan. 24; 109(4):1347-52; and Smith, A M et al., Nucleic Acids Research Can 11, (2010). Further methods and compositions for using barcode technology include those described in U.S. 2016/0060621.

A “transposase” or “tagmentase” (which terms are used synonymously here) means an enzyme that is capable of forming a functional complex with a transposon end-containing composition and catalyzing insertion or transposition of the transposon end-containing composition into the double-stranded target DNA with which it is incubated in an in vitro transposition reaction. Exemplary transposases include but are not limited to modified TN5 transposases that are hyperactive compared to wildtype TN5, for example can have one or more mutations selected from E54K, M56A, or L372P. Transposition works through a “cut-and-paste” mechanism, where the Tn5 excises itself from the donor DNA and inserts into a target sequence, creating a 9-bp duplication of the target (Schaller H. Cold Spring Harb Symp Quant Biol 43: 401-408 (1979); Reznikoff W S., Annu Rev Genet 42: 269-286 (2008)). In current commercial solutions (Nextera DNA kits, Illumina), free synthetic ME adaptors are end-joined to the 5′-end of the target DNA by the transposase.

### DETAILED DESCRIPTION OF THE INVENTION

### Introduction

The inventors have discovered novel methods and compositions for introducing partition-specific barcodes for use in sequencing and other methods. Instead of a single oligonucleotide included on a solid support, the inventors have discovered that a variety of different oligonucleotide sequences can be applied to a single solid support (e.g., a bead), wherein the different oligonucleotides on a single solid support include degenerate nucleotide positions such that the different oligonucleotides on the solid support can each be decoded to indicate a single solid support family identification sequence (e.g., a partition-specific barcode). By introducing different solid supports into different partitions, wherein each solid support has oligonucleotide sequences that are decoded to a different solid support family identification sequence, one can introduce partition-specific labels into partitions. One benefit of this approach over use of a single oligonucleotide per bead, is that an additional unique molecular identifier, a specific sequence unique to each oligonucleotide on the bead, does not need to be added to partition-specific oligonucleotides, thereby allowing for ease in manufacturing of the solid supports. Instead, due to the degeneracy of the oligonucleotide sequences on the solid supports described herein, different oligonucleotides from the same solid support will differ sufficiently to allow for unique count and identification of unique attached sample nucleic acids.

A very simplified version of the invention is discussed in this paragraph for illustrative purposes. Two solid support beads, each introduced into a different partition, can be used to label nucleic acids in the partition in a partition specific-manner. In this example, the oligonucleotide contains a single nucleotide position family identification sequence for solid support #1 that is IUPAC designation W (i.e., A or T) and for solid support #2 that is IUPAC designation S (i.e., G or C). Solid support #1 will have copies (e.g., 500 copies) of an oligonucleotide with T and other copies of an oligonucleotide with A at the barcode position in the oligonucleotide. Solid support #2 will have copies (e.g., 500 copies) of an oligonucleotide with G and other copies of an oligonucleotide with C at the barcode position in the oligonucleotide. The solid supports are introduced into separate partitions (in this simple example two different partitions) and the barcodes are attached to nucleic acids in the partitions. Nucleic acids from the partitions are combined and sequenced. If the barcode position in the sequencing reads is W (i.e., A or T then the nucleic acids came from partition #1 (i.e., the partition containing solid support #1) and if the barcode position is S (i.e., G or C then the nucleic acids came from partition #2 (i.e., the partition containing solid support #2).

There are a variety of iterations for how a solid support barcode can employ degenerate nucleotide positions. A code can be set to define the degeneracy. For ease of use, the IUPAC nucleotide designation for degenerate sequences can be used, though this is not required and other alternatives can also be used. However, in all cases, a code is employed so that the user knows how to decipher the degenerate positions in the oligonucleotide.

Exemplary degenerate IUPAC symbols are as follows:

TABLE 1

| | |
|---|---|
| R | A or G |
| Y | C or T |
| S | G or C |
| W | A or T |
| K | G or T |
| M | A or C |
| B | C or G or T |
| D | A or G or T |
| H | A or C or T |
| V | A or C or G |
| N | any base |

In the above example, a single position in the barcode oligonucleotide indicated the barcode information. However, in other embodiments, 2, 3, 4, 5, 6, 7, 8, 9, 10, 15, 20 or more positions can each provide barcode information. This is useful where a larger number of solid supports and partitions are to be examined. In one example, two nucleotide positions in the oligonucleotide provide information and are degenerate. For example, in some embodiments, two positions (e.g., adjacent positions, though this is not required) are interpreted as follows:

TABLE 2

| X nucleotides | Family identifier |
|---|---|
| WS | T |
| SW | G |
| SS | C |
| WW | A |

In other words, at the two degenerate positions, any sequence representable by “WS” indicates T. Thus AA, AT, TA or TT are all interpreted as “A”. By using multiple nucleotide positions in the oligonucleotide to indicate a single position of the family identification sequence, the number of degenerate sequences that can mean the same nucleotide can be increased. For example, in some embodiments, 2, 3, 4 or more nucleotides of n oligonucleotide can be used to encode a single position of the family identification sequence. Moreover, these can be used in multiples, e.g., 2, 3, 4 sets of 2, 3, 4, or more nucleotides, each set encoding a different position of the family identification sequence.

Thus, in some embodiments, multiple barcode locations in the oligonucleotide can be designated in the code to be degenerate sequences for a single position in the family identification sequence. For example, a barcode can be indicated as XXYXX, where Y is a constant nucleotide (e.g., used to identify the location of the barcode) and each nucleotide X is degenerate where adjacent X pairs indicate one nucleotide. Using Table 2, merely as an example, and using XXYXX as the barcode, the following sequences (among many others) both can be determined to mean the same oligonucleotide family sequence:

| Exemplary | Intermediate | |
|---|---|---|
| XX Y XX sequence | (IUPAC) | Meaning |
| AG Y AG | WS Y WS | T Y T |
| AG Y AC | WS Y WS | T Y T |
| AC Y AC | WS Y WS | T Y T |
| GC Y GC | SS Y SS | C Y C |
| GC Y GA | SS Y SW | C Y G |

In the above example the first three sequences in the oligonucleotide can be deciphered based on IUPAC coding to mean WSYWS and based on Table 2, “WS”=T so this sequence represents “TT”. The remaining sequences above are interpreted in the same manner. Thus, as shown above, various degenerate sequences can be linked up in one oligonucleotide sequence to be decodable to a large number of different solid support family sequences, allowing for a large number of different uniquely-labeled solid supports, each represented by a unique solid support family sequence, which is defined by a number of different degenerate sequences on the solid support.

The location of the degenerate barcode sequence in the oligonucleotide can be determined for example by sequence context. For example, one or a plurality of constant nucleotides can in some embodiments, indicate the position of the degenerate positions. For example, any of a variety of configurations of constant and degenerate positions can be used. In some examples, the barcode sequence can have at least three positions comprising the formula (X)ₙ(Y)ₘ or (Y)ₘ(X)ₙ, wherein X is a degenerate nucleotide, n is 2-50 (e.g., 2-20, 3-20, 4-20, 5-20) and Y is constant, and m is 0-50 (e.g., 1-30, 1-20, 0-10, 1-10). In some embodiments, the sum of n and m is at least three (e.g., 3-50, 3-30, 3-20, 5-50, 10-30, 10-50). In some embodiments, n is 2 and m is 1. For example, the barcode sequence can be or can comprise YXX or XXY.

In some embodiments, the above sequences can be used repeatedly or in combinations to form more complicated barcodes, for example where greater diversity is needed so that more unique solid support family sequences can be employed. As merely some example, the barcodes can comprise one of the following: XₙYₘXₙ, YₘXₙYₘ, XₙYₘXₙYₘXₙ, YₘXₙYₘXₙYₘ, or XₙYₘXₙYₘXₙYₘXₙ, where each n and m or independently selected from the numbering in the above paragraph. For example in some embodiments, x is 2 or 3 or 4 and m is 1. In some embodiments, x is 2 or 3 or 4 and m is 0 or 2.

Each of the above “block” sequences can be used to encode the family identification sequence, or alternatively 2, 3, 4, 5, 6, or more of the sequence blocks can be combined together as separate blocks of the oligonucleotide, which in combination encode the family identification sequence.

The different sequence blocks can be linked together covalently. For example, in some embodiments, the oligonucleotide is a single-stranded nucleic acid comprising the different sequence blocks. The oligonucleotide will generally be single-stranded but in some embodiments can be double-stranded.

In some embodiments, different solid supports are linked to oligonucleotides having sufficiently-different encoded family identification sequences to allow for at least two (e.g., at least 2, at least 3, 2, 3, 4, 5, etc.) differences between any two family identification sequences of the different solid supports. This difference allows for unique identification of barcoded nucleic acids even in the case where for example, one or even two different nucleotides of an oligonucleotide are altered due to amplification or other error introduction, e.g., in sequencing, replication or construction of the oligonucleotides. In another embodiment, the difference allows for unique identification of barcoded nucleic acids even in the case where for example, a sequence is deleted or inserted by error during amplification or other error introduction, e.g., in sequencing, replication or construction of the oligonucleotides. This is exemplified in the Example below.

3′ ends of the oligonucleotides described herein can include a capture sequence, allowing for hybridization of the oligonucleotides to sample molecules (e.g., in the partitions), which can subsequently be extended, ligated, or otherwise attached. Capture sequences can be identical between oligonucleotide or different as desired. Exemplary capture sequences can include, e.g., poly T sequences sufficient to capture poly-adenylated RNA, gene-specific sequences sufficient to enrich for desired sample sequences, random sequences, etc. In some embodiments, the capture sequence is complementary to adaptor sequences, e.g., adaptor sequences introduced by a Tn5 transposases (e.g., via tagmentation).

Following capture of a sample nucleic acid in a partition, the oligonucleotides can be attached to the sample nucleic acids. In case the sample nucleic acids are RNA, a reverse transcriptase can be used. Alternatively, or in combination, a polymerase can be used to extend the oligonucleotide to form a double stranded nucleic acid comprising the sample nucleic acid and the oligonucleotide sequence. Alternatively, ligation or other enzyme activity can link the oligonucleotide to the sample nucleic acid. Once attached, the contents of the partitions, optionally purified, optionally modified with further adaptor or other sequences, can then be sequenced. The partition origin of each sequencing read can be achieved by identification of the family identification sequence (i.e., determine the sequence blocks and use the code to decipher the family identification sequence encoded therein), where sequence reads with the same encoded family identification sequence are interpreted as being from the same partition. As discussed herein, even where certain nucleotide errors occur in the sequence reads for the oligonucleotides, one can distinguish the family identification sequence as being from the most similar family identification sequence because different family identification sequences used are more different. For example, if a sequence read for a family identification sequence has a one nucleotide difference from one expected family identification sequence and two or more differences from all other family identification sequences, then the read can be interpreted as having the one expected family identification sequence.

Nucleic acid samples can be formed into a plurality of separate partitions, e.g., droplets or wells. Any type of partition can be used in the methods described herein. While the method has been exemplified using droplets it should be understood that other types of partitions (e.g., wells) can also be used.

Methods and compositions for partitioning are described, for example, in published patent applications WO 2010/036,352, US 2010/0173,394, US 2011/0092,373, and US 2011/0092,376, the contents of each of which are incorporated herein by reference in the entirety. The plurality of partitions can be in a plurality of emulsion droplets, or a plurality of microwells, etc.

In some embodiments, one or more reagents are added during droplet formation or to the droplets after the droplets are formed. Methods and compositions for delivering reagents to one or more partitions include microfluidic methods as known in the art; droplet or microcapsule combining, coalescing, fusing, bursting, or degrading (e.g., as described in U.S. 2015/0027,892; US 2014/0227,684; WO 2012/149,042; and WO 2014/028,537); droplet injection methods (e.g., as described in WO 2010/151,776); and combinations thereof.

As described herein, the partitions can be picowells, nanowells, or microwells. The partitions can be pico-, nano-, or micro-reaction chambers, such as pico, nano, or microcapsules. The partitions can be pico-, nano-, or micro-channels.

In some embodiments, the partitions are droplets. In some embodiments, a droplet comprises an emulsion composition, i.e., a mixture of immiscible fluids (e.g., water and oil). In some embodiments, a droplet is an aqueous droplet that is surrounded by an immiscible carrier fluid (e.g., oil). In some embodiments, a droplet is an oil droplet that is surrounded by an immiscible carrier fluid (e.g., an aqueous solution). In some embodiments, the droplets described herein are relatively stable and have minimal coalescence between two or more droplets. In some embodiments, less than 0.0001%, 0.0005%, 0.001%, 0.005%, 0.01%, 0.05%, 0.1%, 0.5%, 1%, 2%, 3%, 4%, 5%, 6%, 7%, 8%, 9%, or 10% of droplets generated from a sample coalesce with other droplets. The emulsions can also have limited flocculation, a process by which the dispersed phase comes out of suspension in flakes. In some cases, such stability or minimal coalescence is maintained for up to 4, 6, 8, 10, 12, 24, or 48 hours or more (e.g., at room temperature, or at about 0, 2, 4, 6, 8, 10, or 12° C.). In some embodiments, the droplet is formed by flowing an oil phase through an aqueous sample or reagents.

The oil phase can comprise a fluorinated base oil which can additionally be stabilized by combination with a fluorinated surfactant such as a perfluorinated polyether. In some embodiments, the base oil comprises one or more of a HFE 7500, FC-40, FC-43, FC-70, or another common fluorinated oil. In some embodiments, the oil phase comprises an anionic fluorosurfactant. In some embodiments, the anionic fluorosurfactant is Ammonium Krytox (Krytox-AS), the ammonium salt of Krytox FSH, or a morpholino derivative of Krytox FSH. Krytox-AS can be present at a concentration of about 0.1%, 0.2%, 0.3%, 0.4%, 0.5%, 0.6%, 0.7%, 0.8%, 0.9%, 1.0%, 2.0%, 3.0%, or 4.0% (w/w). In some embodiments, the concentration of Krytox-AS is about 1.8%. In some embodiments, the concentration of Krytox-AS is about 1.62%. Morpholino derivative of Krytox FSH can be present at a concentration of about 0.1%, 0.2%, 0.3%, 0.4%, 0.5%, 0.6%, 0.7%, 0.8%, 0.9%, 1.0%, 2.0%, 3.0%, or 4.0% (w/w). In some embodiments, the concentration of morpholino derivative of Krytox FSH is about 1.8%. In some embodiments, the concentration of morpholino derivative of Krytox FSH is about 1.62%.

In some embodiments, the oil phase further comprises an additive for tuning the oil properties, such as vapor pressure, viscosity, or surface tension. Non-limiting examples include perfluorooctanol and 1H,1H,2H,2H-Perfluorodecanol. In some embodiments, 1H,1H,2H,2H-Perfluorodecanol is added to a concentration of about 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.25%, 1.50%, 1.75%, 2.0%, 2.25%, 2.5%, 2.75%, or 3.0% (w/w). In some embodiments, 1H,1H,2H,2H-Perfluorodecanol is added to a concentration of about 0.18% (w/w).

In some embodiments, the emulsion is formulated to produce highly monodisperse droplets having a liquid-like interfacial film that can be converted by heating into microcapsules having a solid-like interfacial film; such microcapsules can behave as bioreactors able to retain their contents through an incubation period. The conversion to microcapsule form can occur upon heating. For example, such conversion can occur at a temperature of greater than about 40°, 50°, 60°, 70°, 80°, 90°, or 95° C. During the heating process, a fluid or mineral oil overlay can be used to prevent evaporation. Excess continuous phase oil can be removed prior to heating, or left in place. The microcapsules can be resistant to coalescence and/or flocculation across a wide range of thermal and mechanical processing.

Following conversion of droplets into microcapsules, the microcapsules can be stored at about −70°, −20°, 0°, 3°, 4°, 5°, 6°, 7°, 8°, 9°, 10°, 15°, 20°, 25°, 30°, 35°, or 40° C. In some embodiments, these capsules are useful for storage or transport of partition mixtures. For example, samples can be collected at one location, partitioned into droplets containing enzymes, buffers, and/or primers or other probes, optionally one or more polymerization reactions can be performed, the partitions can then be heated to perform microencapsulation, and the microcapsules can be stored or transported for further analysis.

In some embodiments, the sample is partitioned into, or into at least, 500 partitions, 1000 partitions, 2000 partitions, 3000 partitions, 4000 partitions, 5000 partitions, 6000 partitions, 7000 partitions, 8000 partitions, 10,000 partitions, 15,000 partitions, 20,000 partitions, 30,000 partitions, 40,000 partitions, 50,000 partitions, 60,000 partitions, 70,000 partitions, 80,000 partitions, 90,000 partitions, 100,000 partitions, 200,000 partitions, 300,000 partitions, 400,000 partitions, 500,000 partitions, 600,000 partitions, 700,000 partitions, 800,000 partitions, 900,000 partitions, 1,000,000 partitions, 2,000,000 partitions, 3,000,000 partitions, 4,000,000 partitions, 5,000,000 partitions, 10,000,000 partitions, 20,000,000 partitions, 30,000,000 partitions, 40,000,000 partitions, 50,000,000 partitions, 60,000,000 partitions, 70,000,000 partitions, 80,000,000 partitions, 90,000,000 partitions, 100,000,000 partitions, 150,000,000 partitions, or 200,000,000 partitions.

In some embodiments, the droplets that are generated are substantially uniform in shape and/or size. For example, in some embodiments, the droplets are substantially uniform in average diameter. In some embodiments, the droplets that are generated have an average diameter of about 0.001 microns, about 0.005 microns, about 0.01 microns, about 0.05 microns, about 0.1 microns, about 0.5 microns, about 1 microns, about 5 microns, about 10 microns, about 20 microns, about 30 microns, about 40 microns, about 50 microns, about 60 microns, about 70 microns, about 80 microns, about 90 microns, about 100 microns, about 150 microns, about 200 microns, about 300 microns, about 400 microns, about 500 microns, about 600 microns, about 700 microns, about 800 microns, about 900 microns, or about 1000 microns. In some embodiments, the droplets that are generated have an average diameter of less than about 1000 microns, less than about 900 microns, less than about 800 microns, less than about 700 microns, less than about 600 microns, less than about 500 microns, less than about 400 microns, less than about 300 microns, less than about 200 microns, less than about 100 microns, less than about 50 microns, or less than about 25 microns. In some embodiments, the droplets that are generated are non-uniform in shape and/or size.

In some embodiments, the droplets that are generated are substantially uniform in volume. For example, the standard deviation of droplet volume can be less than about 1 picoliter, 5 picoliters, 10 picoliters, 100 picoliters, 1 nL, or less than about 10 nL. In some cases, the standard deviation of droplet volume can be less than about 10-25% of the average droplet volume. In some embodiments, the droplets that are generated have an average volume of about 0.001 nL, about 0.005 nL, about 0.01 nL, about 0.02 nL, about 0.03 nL, about 0.04 nL, about 0.05 nL, about 0.06 nL, about 0.07 nL, about 0.08 nL, about 0.09 nL, about 0.1 nL, about 0.2 nL, about 0.3 nL, about 0.4 nL, about 0.5 nL, about 0.6 nL, about 0.7 nL, about 0.8 nL, about 0.9 nL, about 1 nL, about 1.5 nL, about 2 nL, about 2.5 nL, about 3 nL, about 3.5 nL, about 4 nL, about 4.5 nL, about 5 nL, about 5.5 nL, about 6 nL, about 6.5 nL, about 7 nL, about 7.5 nL, about 8 nL, about 8.5 nL, about 9 nL, about 9.5 nL, about 10 nL, about 11 nL, about 12 nL, about 13 nL, about 14 nL, about 15 nL, about 16 nL, about 17 nL, about 18 nL, about 19 nL, about 20 nL, about 25 nL, about 30 nL, about 35 nL, about 40 nL, about 45 nL, or about 50 nL.

In some embodiments, formation of the droplets results in droplets that comprise the DNA that has been previously treated with the transposase and a first oligonucleotide primer linked to a bead. The term “bead” refers to any solid support that can be in a partition, e.g., a small particle or other solid support. Exemplary beads can include hydrogel beads. In some cases, the hydrogel is in sol form. In some cases, the hydrogel is in gel form. An exemplary hydrogel is an agarose hydrogel. Other hydrogels include, but are not limited to, those described in, e.g., U.S. Pat. Nos. 4,438,258; 6,534,083; 8,008,476; 8,329,763; U.S. Patent Appl. Nos. 2002/0,009,591; 2013/0,022,569; 2013/0,034,592; and International Patent Publication Nos. WO/1997/030092; and WO/2001/049240.

Methods of linking oligonucleotides to beads are described in, e.g., WO 2015/200541. In some embodiments, the oligonucleotide configured to link the hydrogel to the barcode is covalently linked to the hydrogel. Numerous methods for covalently linking an oligonucleotide to one or more hydrogel matrices are known in the art. As but one example, aldehyde derivatized agarose can be covalently linked to a 5′-amine group of a synthetic oligonucleotide.

In some embodiments, the barcode oligonucleotides are attached to a particle or bead. In some embodiments, the particle or bead can be any particle or bead having a solid support surface. Solid supports suitable for particles include controlled pore glass (CPG)(available from Glen Research, Sterling, Va.), oxalyl-controlled pore glass (See, e.g., Alul, et al., Nucleic Acids Research 1991, 19, 1527), TentaGel Support—an aminopolyethyleneglycol derivatized support (See, e.g., Wright, et al., Tetrahedron Letters 1993, 34, 3373), polystyrene, Poros (a copolymer of polystyrene/divinylbenzene), or reversibly cross-linked acrylamide. Many other solid supports are commercially available and amenable to the present invention. In some embodiments, the bead material is a polystyrene resin or poly(methyl methacrylate) (PMMA). The bead material can be metal.

In some embodiments, the particle or bead comprises hydrogel or another similar composition. In some cases, the hydrogel is in sol form. In some cases, the hydrogel is in gel form. An exemplary hydrogel is an agarose hydrogel. Other hydrogels include, but are not limited to, those described in, e.g., U.S. Pat. Nos. 4,438,258; 6,534,083; 8,008,476; 8,329,763; U.S. Patent Appl. Nos. 20020009591; 20130022569; 20130034592; and International Patent Publication Nos. WO1997030092; and WO2001049240. Additional compositions and methods for making and using hydrogels, such as barcoded hydrogels, include those described in, e.g., Klein et al., Cell, 2015 May 21; 161(5):1187-201.

The solid support surface of the bead can be modified to include a linker for attaching barcode oligonucleotides. The linkers may comprise a cleavable moiety. Non-limiting examples of cleavable moieties include a disulfide bond, a dioxyuridine moiety, and a restriction enzyme recognition site.

In some embodiments, the oligonucleotide conjugated to the particle (e.g., a linker) comprises a universal oligonucleotide (universal region) that is directly attached, conjugated, or linked to the solid support surface. In some embodiments, the universal oligonucleotide that is attached to a bead is used for synthesizing a barcode oligonucleotide onto the bead.

In some embodiments, the partitions will include one or a few (e.g., 1, 2, 3, 4) solid supports (e.g., beads) per partition (e.g., as occurs in a Poisson distribution), where each solid support is linked to an oligonucleotide primer having a free 3′ end. The oligonucleotide primer will have an underlying family solid-support-specific barcode and a 3′ end that is complementary a target sequence, which could be as non-limiting examples an adaptor introduced by a tagmentase, a polyA, a specific gene sequence, or a random sequence. The barcode can be continuous or discontinuous, i.e., broken up by other nucleotides.

In some embodiments, the 3′ end will be at least 50% complementary (e.g., at least 60%, 70%, 80%, 90% or 100%) complementary (such that they hybridize) to an adaptor sequence. In some embodiments, at least the 3′-most 6, 7, 8, 9, 10, 11, 12, 13, 14, 15, 16, 17, 18, 19, or 20 of the oligonucleotide are at least 50% complementary (e.g., at least 60%, 70%, 80%, 90% or 100%) complementary to a sequence in the adaptor. The adaptor sequence in some embodiments comprises GACGCTGCCGACGA (A14; SEQ ID NO:1) or CCGAGCCCACGAGAC (B15; SEQ ID NO:2).

In some embodiments, the oligonucleotide associated with the solid support further comprises a universal or other additional sequence to assist with downstream manipulation or sequencing of the amplicon. For example, when Illumina-based sequencing is used the oligonucleotide primer can have a 5′ P5 or P7 sequence (optionally with the second oligonucleotide primer having the other of the two sequences).

The oligonucleotide can be associated with the solid support by a reversible (e.g., releasable) linker. In some embodiments, the oligonucleotide is associated with the solid support by being contained by or on the solid support, for example where the solid support is a hydrogel or other dissolvable solid support. Optionally, the oligonucleotide primer comprises a restriction or cleavage site to remove the oligonucleotide primer from the solid support when desired. In some cases, the oligonucleotide primer is attached to a solid support (e.g., bead) through a disulfide linkage (e.g., through a disulfide bond between a sulfide of the solid support and a sulfide covalently attached to the 5′ or 3′ end, or an intervening nucleic acid, of the oligonucleotide). In such cases, the oligonucleotide can be cleaved from the solid support by contacting the solid support with a reducing agent such as a thiol or phosphine reagent, including but not limited to a beta mercaptoethanol, dithiothreitol (DTT), or tris(2-carboxyethyl)phosphine (TCEP). In some embodiments, the oligonucleotide can be covalently attached to the building block (polymer) of a solid support (e.g. polyacrylamide), of which the polymer cross-linkage is through disulfide linkage. An exemplary polyacrylaminde type would be sensitive (dissolvable when exposed) to reducing agents is Bac (N,N′-Bis(acryloyl)cystamine). In these embodiments, the solid-support itself becomes cleavable/dissolvable in the presence of reducing agent, and the oligonucleotide attached to the polymer can be released through the cleavage/dissolution of the solid support.

In some embodiments, once the nucleic acid sample is in the partitions with the solid support-linked first oligonucleotide primer but prior to hybridization, the oligonucleotide primer is cleaved from the bead prior to amplification. To the extent more than one bead (and thus bead-specific barcode via the oligonucleotide primer) is introduced into a droplet, deconvolution can be used to orient sequence data from a particular bead to that bead. One approach for deconvoluting which beads are present together in a single partition is to provide partitions with substrates comprising barcode sequences for generating a unique combination of sequences for beads in a particular partition, such that upon their sequence analysis (e.g., by next-generation sequencing), the beads are virtually linked. See, e.g., PCT Application WO2017/120531.

In some embodiments, the partitions can further include a second oligonucleotide primer that functions as a reverse primer in combination with the oligonucleotide primer associated with the solid support as described above. In some embodiments, the 3′ end of the second oligonucleotide primer is at least 50% complementary (e.g., at least 60%, 70%, 80%, 90% or 100%) complementary to a 3′ single-stranded portion of an oligonucleotide adaptor ligated to a DNA fragment. In some embodiments, the 3′ end of the second oligonucleotide primer will be complementary to the entire adaptor sequence. In some embodiments, at least the 3′-most 6, 7, 8, 9, 10, 11, 12, 13, 14, 15, 16, 17, 18, 19, or 20 of the second oligonucleotide primer are complementary to a sequence in the adaptor. In some embodiments, the second oligonucleotide primer comprises a barcode sequence, which for example can be of the same length as listed above for the barcode of the oligonucleotide primer described elsewhere herein. In some embodiments, the barcode includes an index barcode, e.g., a sample barcode, e.g., Illumina i7 or i5 sequences.

In some embodiments, where information about a haploid genome is desired, the sample is DNA in the partitions maintained such that contiguity between fragments created by a transposase is maintained. This can be achieved for example, by selecting conditions such that a transposase cleaves genomic DNA (e.g., in a chromatin-specific matter) but does not release from the DNA, and thus forms a bridge linking DNA segments that have the same relationship (haplotype) as occurred in the genomic DNA. For example, transposase has been observed to remain bound to DNA until a detergent such as SDS is added to the reaction (Amini et al. Nature Genetics 46(12):1343-1349).

Any method of nucleotide sequencing can be used as desired so long as at least some of the DNA segments sequence and the barcode sequence is determined. Methods for high throughput sequencing and genotyping are known in the art. For example, such sequencing technologies include, but are not limited to, pyrosequencing, sequencing-by-ligation, single molecule sequencing, sequence-by-synthesis (SBS), massive parallel clonal, massive parallel single molecule SBS, massive parallel single molecule real-time, massive parallel single molecule real-time nanopore technology, etc. Morozova and Marra provide a review of some such technologies in Genomics, 92: 255 (2008), herein incorporated by reference in its entirety.

Exemplary DNA sequencing techniques include fluorescence-based sequencing methodologies (See, e.g., Birren et al., Genome Analysis: Analyzing DNA, 1, Cold Spring Harbor, N.Y.; herein incorporated by reference in its entirety). In some embodiments, automated sequencing techniques understood in that art are utilized. In some embodiments, the present technology provides parallel sequencing of partitioned amplicons (PCT Publication No. WO 2006/0,841,32, herein incorporated by reference in its entirety). In some embodiments, DNA sequencing is achieved by parallel oligonucleotide extension (See, e.g., U.S. Pat. Nos. 5,750,341; and 6,306,597, both of which are herein incorporated by reference in their entireties). Additional examples of sequencing techniques include the Church polony technology (Mitra et al., 2003, Analytical Biochemistry 320, 55-65; Shendure et al., 2005 Science 309, 1728-1732; and U.S. Pat. Nos. 6,432,360; 6,485,944; 6,511,803; herein incorporated by reference in their entireties), the 454 picotiter pyrosequencing technology (Margulies et al., 2005 Nature 437, 376-380; U.S. Publication No. 2005/0130173; herein incorporated by reference in their entireties), the Solexa single base addition technology (Bennett et al., 2005, Pharmacogenomics, 6, 373-382; U.S. Pat. Nos. 6,787,308; and 6,833,246; herein incorporated by reference in their entireties), the Lynx massively parallel signature sequencing technology (Brenner et al. (2000). Nat. Biotechnol. 18:630-634; U.S. Pat. Nos. 5,695,934; 5,714,330; herein incorporated by reference in their entireties), and the Adessi PCR colony technology (Adessi et al. (2000). Nucleic Acid Res. 28, E87; WO 2000/018957; herein incorporated by reference in its entirety).

Typically, high throughput sequencing methods share the common feature of massively parallel, high-throughput strategies, with the goal of lower costs in comparison to older sequencing methods (See, e.g., Voelkerding et al., Clinical Chem., 55: 641-658, 2009; MacLean et al., Nature Rev. Microbiol., 7:287-296; each herein incorporated by reference in their entirety). Such methods can be broadly divided into those that typically use template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and platforms commercialized by VisiGen, Oxford Nanopore Technologies Ltd., Life Technologies/Ion Torrent, and Pacific Biosciences, respectively.

In pyrosequencing (Voelkerding et al., Clinical Chem., 55: 641-658, 2009; MacLean et al., Nature Rev. Microbial., 7:287-296; U.S. Pat. Nos. 6,210,891; and 6,258,568; each herein incorporated by reference in its entirety), template DNA is fragmented, end-repaired, ligated to adaptors, and clonally amplified in-situ by capturing single template molecules with beads bearing oligonucleotides complementary to the adaptors. Each bead bearing a single template type is compartmentalized into a water-in-oil microvesicle, and the template is clonally amplified using a technique referred to as emulsion PCR. The emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 10⁶ sequence reads can be achieved, resulting in up to 500 million base pairs (Mb) of sequence.

In the Solexa/Illumina platform (Voelkerding et al., Clinical Chem., 55. 641-658, 2009; MacLean et al., Nature Rev. Microbial., 7:287-296; U.S. Pat. Nos. 6,833,246; 7,115,400; and 6,969,488; each herein incorporated by reference in its entirety), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A-addition facilitates addition of T-overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluor and block removed prior to the next cycle of dNTP addition. Sequence read length ranges from 36 nucleotides to over 50 nucleotides, with overall output exceeding 1 billion nucleotide pairs per analytical run.

Sequencing nucleic acid molecules using SOLiD technology (Voelkerding et al., Clinical Chem., 55: 641-658, 2009; MacLean et al., Nature Rev. Microbial., 7:287-296; U.S. Pat. Nos. 5,912,148; and 6,130,073; each herein incorporated by reference in their entirety) also involves fragmentation of the template, ligation to oligonucleotide adaptors, attachment to beads, and clonal amplification by emulsion PCR. Following this, beads bearing template are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color, and thus identity of each probe, corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, nanopore sequencing is employed (See, e.g., Astier et al., J. Am. Chem. Soc. 2006 Feb. 8; 128(5)1705-10, herein incorporated by reference). The theory behind nanopore sequencing has to do with what occurs when a nanopore is immersed in a conducting fluid and a potential (voltage) is applied across it. Under these conditions a slight electric current due to conduction of ions through the nanopore can be observed, and the amount of current is exceedingly sensitive to the size of the nanopore. As each base of a nucleic acid passes through the nanopore, this causes a change in the magnitude of the current through the nanopore that is distinct for each of the four bases, thereby allowing the sequence of the DNA molecule to be determined.

In certain embodiments, HeliScope by Helicos BioSciences is employed (Voelkerding et al., Clinical Chem., 55. 641-658, 2009; MacLean et al., Nature Rev. Microbial, 7:287-296; U.S. Pat. Nos. 7,169,560; 7,282,337; 7,482,120; 7,501,245; 6,818,395; 6,911,345; and 7,501,245; each herein incorporated by reference in their entirety). Template DNA is fragmented and polyadenylated at the 3′ end, with the final adenosine bearing a fluorescent label. Denatured polyadenylated template fragments are ligated to poly(dT) oligonucleotides on the surface of a flow cell. Initial physical locations of captured template molecules are recorded by a CCD camera, and then label is cleaved and washed away. Sequencing is achieved by addition of polymerase and serial addition of fluorescently-labeled dNTP reagents. Incorporation events result in fluor signal corresponding to the dNTP, and signal is captured by a CCD camera before each round of dNTP addition. Sequence read length ranges from 25-50 nucleotides, with overall output exceeding 1 billion nucleotide pairs per analytical run.

The Ion Torrent technology is a method of DNA sequencing based on the detection of hydrogen ions that are released during the polymerization of DNA (See, e.g., Science 327(5970): 1190 (2010); U.S. Pat. Appl. Pub. Nos. 2009/0026082; 2009/0127589; 2010/0301398; 2010/0197507; 2010/0188073; and 2010/0137143, incorporated by reference in their entireties for all purposes). A microwell contains a template DNA strand to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers the hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per base accuracy of the Ion Torrent sequencer is ^{˜}99.6% for 50 base reads, with ^{˜}100 Mb generated per run. The read-length is 100 base pairs. The accuracy for homopolymer repeats of 5 repeats in length is ^{˜}98%. The benefits of ion semiconductor sequencing are rapid sequencing speed and low upfront and operating costs.

Another exemplary nucleic acid sequencing approach that may be adapted for use with the present invention was developed by Stratos Genomics, Inc. and involves the use of Xpandomers. This sequencing process typically includes providing a daughter strand produced by a template-directed synthesis. The daughter strand generally includes a plurality of subunits coupled in a sequence corresponding to a contiguous nucleotide sequence of all or a portion of a target nucleic acid in which the individual subunits comprise a tether, at least one probe or nucleobase residue, and at least one selectively cleavable bond. The selectively cleavable bond(s) is/are cleaved to yield an Xpandomer of a length longer than the plurality of the subunits of the daughter strand. The Xpandomer typically includes the tethers and reporter elements for parsing genetic information in a sequence corresponding to the contiguous nucleotide sequence of all or a portion of the target nucleic acid. Reporter elements of the Xpandomer are then detected. Additional details relating to Xpandomer-based approaches are described in, for example, U.S. Pat. Pub No. 2009/0035777, which is incorporated herein in its entirety.

Other single molecule sequencing methods include real-time sequencing by synthesis using a VisiGen platform (Voelkerding et al., Clinical Chem., 55: 641-58, 2009; U.S. Pat. No. 7,329,492; and U.S. patent application Ser. Nos. 11/671,956; and 11/781,166; each herein incorporated by reference in their entirety) in which immobilized, primed DNA template is subjected to strand extension using a fluorescently-modified polymerase and florescent acceptor molecules, resulting in detectible fluorescence resonance energy transfer (FRET) upon nucleotide addition.

Another real-time single molecule sequencing system developed by Pacific Biosciences (Voelkerding et al., Clinical Chem., 55. 641-658, 2009; MacLean et al., Nature Rev. Microbiol., 7:287-296; U.S. Pat. Nos. 7,170,050; 7,302,146; 7,313,308; and 7,476,503; all of which are herein incorporated by reference) utilizes reaction wells 50-100 nm in diameter and encompassing a reaction volume of approximately 20 zeptoliters (10⁻²¹ L). Sequencing reactions are performed using immobilized template, modified phi29 DNA polymerase, and high local concentrations of fluorescently labeled dNTPs. High local concentrations and continuous reaction conditions allow incorporation events to be captured in real time by fluor signal detection using laser excitation, an optical waveguide, and a CCD camera.

In certain embodiments, the single molecule real time (SMRT) DNA sequencing methods using zero-mode waveguides (ZMWs) developed by Pacific Biosciences, or similar methods, are employed. With this technology, DNA sequencing is performed on SMRT chips, each containing thousands of zero-mode waveguides (ZMWs). A ZMW is a hole, tens of nanometers in diameter, fabricated in a 100 nm metal film deposited on a silicon dioxide substrate. Each ZMW becomes a nanophotonic visualization chamber providing a detection volume of just 20 zeptoliters (10⁻²¹ L). At this volume, the activity of a single molecule can be detected amongst a background of thousands of labeled nucleotides. The ZMW provides a window for watching DNA polymerase as it performs sequencing by synthesis. Within each chamber, a single DNA polymerase molecule is attached to the bottom surface such that it permanently resides within the detection volume. Phospholinked nucleotides, each type labeled with a different colored fluorophore, are then introduced into the reaction solution at high concentrations which promote enzyme speed, accuracy, and processivity. Due to the small size of the ZMW, even at these high concentrations, the detection volume is occupied by nucleotides only a small fraction of the time. In addition, visits to the detection volume are fast, lasting only a few microseconds, due to the very small distance that diffusion has to carry the nucleotides. The result is a very low background.

Processes and systems for such real time sequencing that may be adapted for use with the methods described herein are described in, for example, U.S. Pat. Nos. 7,405,281; 7,315,019; 7,313,308; 7,302,146; and 7,170,050; and U.S. Pat. Pub. Nos. 2008/0212960; 2008/0206764; 2008/0199932; 2008/0199874; 2008/0176769; 2008/0176316; 2008/0176241; 2008/0165346; 2008/0160531; 2008/0157005; 2008/0153100; 2008/0153095; 2008/0152281; 2008/0152280; 2008/0145278; 2008/0128627; 2008/0108082; 2008/0095488; 2008/0080059; 2008/0050747; 2008/0032301; 2008/0030628; 2008/0009007; 2007/0238679; 2007/0231804; 2007/0206187; 2007/0196846; 2007/0188750; 2007/0161017; 2007/0141598; 2007/0134128; 2007/0128133; 2007/0077564; 2007/0072196; and 2007/0036511; and Korlach et al. (2008) “Selective aluminum passivation for targeted immobilization of single DNA polymerase molecules in zero-mode waveguide nanostructures” PNAS 105(4): 1176-81, all of which are herein incorporated by reference in their entireties.

As noted above, upon completion of sequencing, sequences can be sorted by same underlying family barcode, wherein sequences having the same barcode came from the same partition. In view of the degeneracy of the family identification sequences, to the extent certain errors occur in replication of barcodes, the sequence reads can nevertheless be accurately interpreted into the origin family identification sequence because of the sequences tolerances for a certain number of errors and in view of the known family identification sequences used in the first place.

It is understood that the examples and embodiments described herein are for illustrative purposes only and that various modifications or changes in light thereof will be suggested to persons skilled in the art and are to be included within the spirit and purview of this application and scope of the appended claims. All publications, sequence accession numbers, patents, and patent applications cited herein are hereby incorporated by reference in their entirety for all purposes.

### EXAMPLE

### Example 1

Examples of the code structure. Oligonucleotide members of a family are related by code of sequence in (X)ₙ(Y)ₘ scheme. Code (X) and (Y) can be degenerate or constant nucleotide/polynucleotide sequences of length “n” and “m”, respectively. Example 1 and 2 illustrate (W)₂(A)₁ and (W)₃(S)₁ family code and their corresponding member sequences expanded from the family code.

Exemplary decoding of oligonucleotide sequences into family identification sequences: (X)ₙ(Y)ₘ, if X=degenerate base W; n=2; Y=constant base A; m=1

Family code: W,W,A
(According to IUPAC degeneracy rule: W=A/T)
All possible “member” sequence combinations: A/T,A/T,A

### A,A,A

### A,T,A

### T,A,A

### T,T,A

A barcode oligonucleotide-conjugated bead can be generated in which the oligonucleotides each include a barcode selected from AAA, ATA, TAA, and TTA. The bead will be conjugated to different oligonucleotides having AAA, ATA, TAA, or TTA such that the bead is linked to some (e.g., substantially equal numbers of) oligonucleotides having the different listed barcodes. The bead can then be linked in a partition (e.g., droplet) to sample polynucleotides in the partition to form tagged sample polynucleotides. Different sample polynucleotides in the partition will receive different barcoded oligonucleotides but all barcodes will encode the same family barcode. Tagged sample polynucleotides can subsequently be mixed with tagged sample polynucleotides from different partitions that have been tagged with different barcodes. The mixture can be nucleotide sequenced. Sequencing reads will contain barcode sequences and the barcodes can be groups by encoded family barcode (e.g., by a computer) applying a code such as described above to the barcode sequence. Sequence reads for example that include the encoded WWA family barcode will all be from the same partition.

### Example 2

(X)ₙ(Y)ₘ, if X=degenerate base W; n=3; Y=degenerate base S; m=1

Family code: W,W,W,S
(According to IUPAC degeneracy rule: W=A/T; S=G/C)
All possible “member” sequence combinations: A/T,A/T,A/T,G/C

### A,A,A,G

### A,A,T,G

### A,T,A,G

### A,T,T,G

### T,A,A,G

### T,A,T,G

### T,T,A,G

### T,T,T,G

### A,A,A,C

### A,A,T,C

### A,T,A,C

### A,T,T,C

### T,A,A,C

### T,A,T,C

### T,T,A,C

### T,T,T,C

A barcode oligonucleotide-conjugated bead can be generated in which the oligonucleotides each include a barcode selected from AAAG, AATG, ATAG, ATTG, TAAG, TATG, TTAG, TTTG, AAAC, AATC, ATAC, ATTC, TAAC, TATC, TTAC, and TTTC. The bead will be conjugated to different oligonucleotides having at least some of AAAG, AATG, ATAG, ATTG, TAAG, TATG, TTAG, TTTG, AAAC, AATC, ATAC, ATTC, TAAC, TATC, TTAC, or TTTC such that the bead is linked to some (e.g., substantially equal numbers of) oligonucleotides having at least some or all of the different listed barcodes. The bead can then be linked in a partition (e.g., droplet) to sample polynucleotides in the partition to form tagged sample polynucleotides. Different sample polynucleotides in the partition will receive different barcoded oligonucleotides but all barcodes will encode the same family barcode. Tagged sample polynucleotides can subsequently be mixed with tagged sample polynucleotides from different partitions that have been tagged with different barcodes. The mixture can be nucleotide sequenced. Sequencing reads will contain barcode sequences and the barcodes can be groups by encoded family barcode (e.g., by a computer) applying a code such as described above to the barcode sequence. Sequence reads for example that include the encoded WWWS family barcode will all be from the same partition.

### Example 3

An example of self-correction feature of barcode design. Among a whitelist of two barcode sequences (e.g. GGACG and GGTCT) of >1 Hamming Distance apart, the original barcode sequence “GGACG” is coded by wobble base substitution according to the conversion table under (X)m(Y)n scheme to “SWSSWSASSWG”. If an error arises during sequencing and results in an ambiguous base calling at position-1 as shown, the ambiguous base of coded-barcode can be self-corrected by collapsing wobble sequence calculating the Hamming/Levensthein Distance against known barcode sequences.

1. GGACG—Original barcode
2. (SWS)(SWS)(A)(SSW)(G)—Coded under (X)ₘ(Y)ₙ scheme
Possible conversion table:
   WSW=base A
   SWS=base G
   SSW=base C
   WWS=base T
   3. SWSSWSASSWG—Coded-barcode sequence (family code)
   If the sequencer were to fail a base call at position 1:
   4. NWSSWSASSWG—Read with substitution error at position 1
   5. (NWS)GACG—Collapse wobble sequences to nucleotide base
Ambiguous wobble sequences due to N at 1^{st} position
Correct the error by expanding the NWS sequence to possible barcode
N can only be S or W according to arbitrary conversion table
NWS can be converted to SWS or WWS, and further collapsed to G or T.
   6. (G)GACG or (T)GACG—Possible barcode sequence
   7. TGACG—(1 Hamming Distance from GGACG & not <=1 edit from any other block)
   GGACG—(0 Hamming Distance from GGACG)
   9. GGACG—Final barcode sequence called based on shortest Hamming distance

### Example 4

An example of self-correction feature of barcode design. Among a whitelist of two barcode sequences (e.g. GGACG and GGTCT) of >1 Hamming Distance apart, the original barcode sequence “GGACG” is coded by wobble base substitution according to the conversion table under (X)m(Y)n scheme to “SWSSWSASSWG”. If errors arise during sequencing and result in 2 ambiguous base calling at position-1 and 6 as shown, the ambiguous bases of coded-barcode can be self-corrected by collapsing wobble sequence and then calculating the Hamming/Levensthein Distance against know barcode sequences.

1. GGACG—Original barcode
2. (SWS)(SWS)(A)(SSW)(G)—Coded under (X)m(Y)n scheme
Arbitrary conversion table:
   WSW=base A
   SWS=base G
   SSW=base C
   WWS=base T
   3. SWSSWSASSWG—Coded-barcode sequence (family code)
   If the sequencer were to fail a base call at positon 1:
   4. NWSSWNASSWG—Read with substitution error at position 1 and 6
   5. (NWS)(SWN)ACG—Collapse wobble sequences to nucleotide base
Ambiguous wobble sequences due to N at both 1^{st} and 6^{th} position
Correct the error by expanding the NWS and SWN sequence to possible barcode
NWS can be converted to SWS or WWS, and further collapsed to G or T.
SWN can be converted to SWS, and further collapsed to G
   6. (G)GACG or (T)GACG—Possible barcode sequence
   7. TGACG—(1 Hamming Distance from GGACG & not <=1 edit from any other block)
   GGACG—(0 Hamming Distance from GGACG)
   9. GGACG—Final barcode sequence called based on shortest Hamming distance

### Example 5

This example further illustrates the error tolerance improvement of (X)ₘ(Y)ₙ design scheme as compared to the conventional barcode design. For an example whitelist of two barcode sequences, CAGGCGG and GGTCTGA, if a conventional design defines an uncallable barcode as >1 Hamming Distance from any designed code sequences, this means either sequence can tolerates an error of only one edit (e.g. CAGGCGG versus NAGGCGG) but not two or more edits (e.g. CAGGCGG versus NNGGCGG or NNNGCGG). Thus, this setup tolerates only 1/7 (or ˜14%) of the barcode to mutate before it is deemed uncallable.

Using the (X)ₘ(Y)ₙ design scheme to expand the same code sequence, for instance CAGGCGG to SSASWGSSGSW, allows for greater error tolerance depending on where those errors occur. If no more than one “constant, Y” base is mutated, the expanded barcode becomes quite robust to mutations as illustrated below:

1. CAGGCGG—Original barcode sequence
2. SSASWGSSGSW—Code expanded under (X)ₘ(Y)ₙ design scheme by using Table 2 conversion table
3. NSNNWGNSGNW—Mutate this to convert the first position of every wobble block (underlined) AND a mutation in the first “constant” base (bold). The string NSNNWGNSGNW has an N in the first position of every wobble block AND a mutation in the first “constant” base. Therefore, 5 of the 11 bases in the barcode are mutated (or ˜45%).

Using Table 2 as code for conversion, the ambiguous sequence (i.e. NSNNWGNSGNW) is collapsed to all possible sequences as below:

### Possible seq #1=ANTGAGT

### Possible seq #2=CNTGAGT

### Possible seq #3=ANGGAGT

### Possible seq #4=CNGGAGT

### Possible seq #5=ANTGCGT

### Possible seq #6=CNTGCGT

### Possible seq #7=ANGGCGT

### Possible seq #8=CNGGCGT

### Possible seq #9=ANTGAGG

### Possible seq #10=CNTGAGG

### Possible seq #11=ANGGAGG

### Possible seq #12=CNGGAGG

### Possible seq #13=ANTGCGG

### Possible seq #14=CNTGCGG

### Possible seq #15=ANGGCGG

Possible seq #16=CNGGCGG←This is the only one within 1 edit distance of CAGGCGG.

As a result, even NSNNWGNSGNW, which has 5 Ns, would still be possible to call as CAGGCGG following the original barcode design rule. The design scheme described herein improves error tolerance from 14% to 45% without changing the design rule of deeming uncallable barcode as >1 Hamming Distance. This example also demonstrates that more the degenerate bases of (X)ₘ(Y)ₙ design scheme, greater the error tolerance.

### Example 6

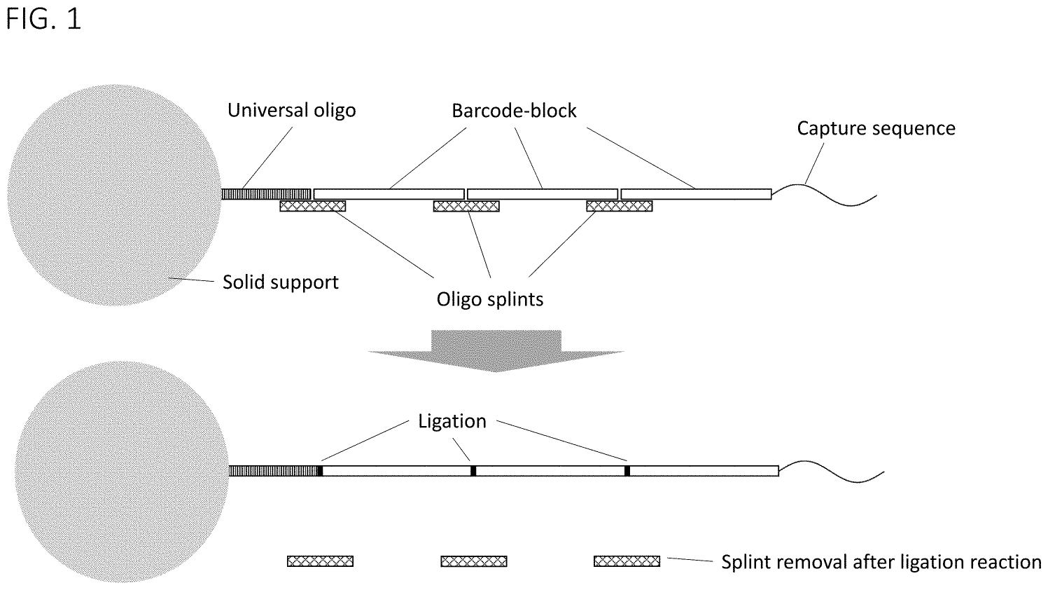

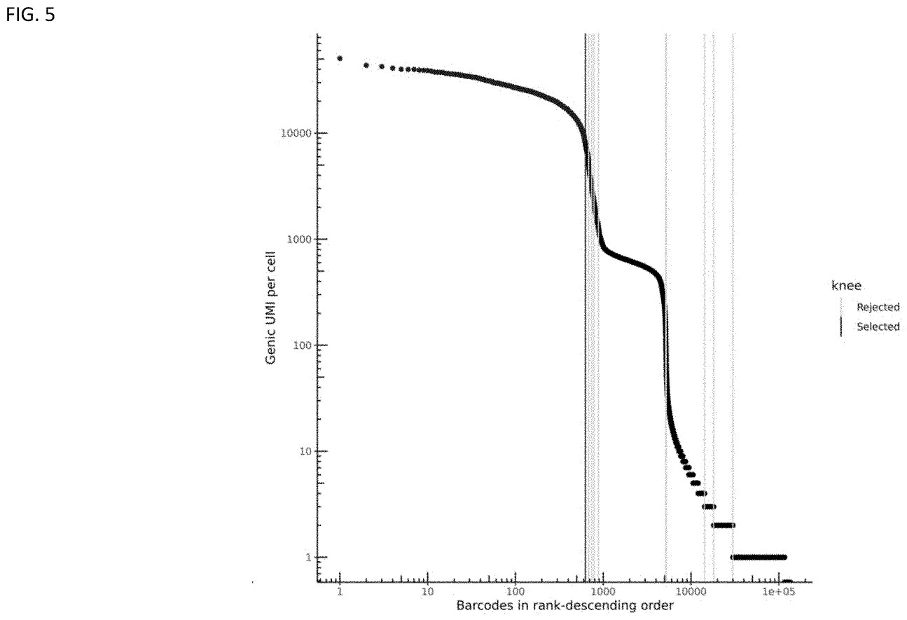

Full-length barcode oligonucleotides of the (X)ₘ(Y)ₙ design scheme were constructed on beads as illustrated in FIG. 1. Cells and barcode oligonucleotides-conjugated beads were encapsulated in water-in-oil droplet partitions. Barcode tagged cDNAs were then synthesized in each partition after cell-lysis and RNA transcripts capturing by the barcode oligonucleotides released in the same partition. Droplets were then broken and the second-strand cDNA synthesis was performed in bulk solution to make double-stranded cDNA library. Following the Illumina Nextera tagmentation library preparation, a PCR was then performed to amplify the double-stranded cDNA library by using Illumina sequencing adapters. The amplified single-cell RNA-seq libraries were then sequenced on Illumina sequencers. The single-cell deconvolution and 3′-tagged transcriptome gene profiling analysis was carried out by using bioinformatics. Valid barcodes sequence was deconvoluted according to the (X)ₘ(Y)ₙ design scheme, and the number of single-cells was then determined by single-cell knee-calling analysis as shown in FIG. 5.

### Example 7

Two builds of beads using the dimer barcode were used:

AAGCAGTGGTATCAAGCAGAGTACndndndndn[0|G|CG|TCC|HDCG]ATGACTACACndndndndnTC AGGACATCndndndndnTTTTTTTTTTTTTTTTTTTTTTTTTTTTTT (SEQ ID NO: 3), where d is the wobble sequence—in this case two nucleotides per occurrence of “d”—used to identify a family (see below table and 2 specific examples below) and n is a single nucleotide; and a reference whitelist barcode bead with CBC and UMI and a bead containing a random CBC and UMI (Macosko (Drop-Seq) Lot #012819c) were compared for their ability to capture mRNA, be processed to generate sequence libraries, and subsequently have the libraries analyzed by an automated pipeline that parses the resulting sequencing data to associate the resulting sequences based on the bead barcode and distinguishes duplicate sequences due to duplicate mRNAs from workflow duplicates based on the unique elements of the different barcodes (e.g., dimer code or UMI depending on bead type). Each bead thus has several members that belong to the same family and thus have several different options for the wobble barcode oligonucleotides.

The 384 family identifiers for the three ndndndndn blocks are as follows, with all 384 being used per each of the 3 blocks, where WS=A, SW=G, SS=C, WW=T:

| | family code | family identifier |
|---|---|---|
| 1 | AWSAWSAWSAWSA | AAAAAAAAA |
| 2 | AWSAWSASWGSWG | AAAAAGGGG |
| 3 | AWSASWGWSASWG | AAAGGAAGG |
| 4 | AWSASWGSWGWSA | AAAGGGGAA |
| 5 | AWSCWSCWSCWSC | AACACACAC |
| 6 | AWSCSWTSSAWWG | AACGTCATG |
| 7 | AWSCSWTSWTWSC | AACGTGTAC |
| 8 | AWSCWWGWSCWWG | AACTGACTG |
| 9 | AWSCWWGSWTSSA | AACTGGTCA |
| 10 | AWSCWWGWWGWSC | AACTGTGAC |
| 11 | AWSGWWCWSGWWC | AAGTCAGTC |
| 12 | AWSTWSTSWCSWC | AATATGCGC |
| 13 | AWSTSWCWSTSWC | AATGCATGC |
| 14 | ASSASSAWSCWSC | ACACAACAC |
| 15 | ASSASSASSASSA | ACACACACA |
| 16 | ASSASSAWWGWWG | ACACATGTG |
| 17 | ASSAWWGSWTWSC | ACATGGTAC |
| 18 | ASSGWSTWWASWC | ACGATTAGC |
| 19 | ASSTSSTWWCWWC | ACTCTTCTC |
| 20 | ASSTSWAWSGWWC | ACTGAAGTC |
| 21 | ASSTWWCSSTWWC | ACTTCCTTC |
| 22 | ASWASSTWSGWWC | AGACTAGTC |
| 23 | ASWCWSTWSTSWC | AGCATATGC |
| 24 | ASWCSWCSWCSWC | AGCGCGCGC |
| 25 | ASWGWSASWGWSA | AGGAAGGAA |
| 26 | ASWGSWGSWGSWG | AGGGGGGGG |
| 27 | ASWTSSAWSCWWG | AGTCAACTG |
| 28 | ASWTSSASWTSSA | AGTCAGTCA |
| 29 | ASWTSSAWWGWSC | AGTCATGAC |
| 30 | ASWTSWTWSCWSC | AGTGTACAC |
| 31 | CWSAWSASSAWWG | CAAAACATG |
| 32 | CWSAWSASWTWSC | CAAAAGTAC |
| 33 | CWSASWGWSCWSC | CAAGGACAC |
| 34 | CWSAWWTSSAWSC | CAATTCAAC |
| 35 | CWSCWSCWSASWG | CACACAAGG |
| 36 | CWSCWSCSWGWSA | CACACGGAA |
| 37 | CWSCSWTWSAWSA | CACGTAAAA |
| 38 | CWSGWSGWSTSWC | CAGAGATGC |
| 39 | CWSTSSGWSGWWC | CATCGAGTC |
| 40 | CWWASSGWWCWWC | CTACGTCTC |
| 41 | CWWASWCWSGWWC | CTAGCAGTC |
| 42 | CWWCWSGWWASWC | CTCAGTAGC |
| 43 | CWWCSSTSWCSWC | CTCCTGCGC |
| 44 | CWWCWWCWSTSWC | CTCTCATGC |
| 45 | CWWGWSCWWTSWG | CTGACTTGG |
| 46 | CWWGWWGSWGWSA | CTGTGGGAA |
| 47 | CWWTSWGWSCWWG | CTTGGACTG |
| 48 | CWWTSWGSWTSSA | CTTGGGTCA |
| 49 | CWWTSWGWWGWSC | CTTGGTGAC |
| 50 | GWSAWSAWSGWWC | GAAAAAGTC |
| 51 | GWSCWWGSWCSWC | GACTGGCGC |
| 52 | GWSGWSGWWGWWG | GAGAGTGTG |
| 53 | GWSGWWCSWGSWG | GAGTCGGGG |
| 54 | GWSTWSTSWTSSA | GATATGTCA |
| 55 | GWSTSWCSSAWSC | GATGCCAAC |
| 56 | GWSTWWASSASSA | GATTACACA |
| 57 | GSWAWSGWWTSWG | GGAAGTTGG |
| 58 | GSWASSTWSAWSA | GGACTAAAA |
| 59 | GSWASSTSWGSWG | GGACTGGGG |
| 60 | GSWCSSGWSCWSC | GGCCGACAC |
| 61 | GSWCSWCSWTSSA | GGCGCGTCA |
| 62 | GSWCSWCWWGWSC | GGCGCTGAC |
| 63 | GSWCWWASSAWWG | GGCTACATG |
| 64 | GSWCWWASWTWSC | GGCTAGTAC |
| 65 | GSWGSWGWSGWWC | GGGGGAGTC |
| 66 | GSWGWWTSSTWWC | GGGTTCTTC |
| 67 | GSWTWSCWWASWC | GGTACTAGC |
| 68 | GSWTSSASWCSWC | GGTCAGCGC |
| 69 | GWWASWCSWTWSC | GTAGCGTAC |
| 70 | GWWCSWAWSASWG | GTCGAAAGG |
| 71 | GWWCSWASWGWSA | GTCGAGGAA |
| 72 | GWWGSWTWSTSWC | GTGGTATGC |
| 73 | TWSGWWCSSAWWG | TAGTCCATG |
| 74 | TWSTSSGWSAWSA | TATCGAAAA |
| 75 | TWSTSWCWWGWWG | TATGCTGTG |
| 76 | TWSTWWASWGWSA | TATTAGGAA |
| 77 | TSSASWTSSTWWC | TCAGTCTTC |
| 78 | TSSGWSTSWGSWG | TCGATGGGG |
| 79 | TSSTWSGWSCWSC | TCTAGACAC |
| 80 | TSSTSWASSAWWG | TCTGACATG |
| 81 | TSSTWWCWSCWWG | TCTTCACTG |
| 82 | TSSTWWCSWTSSA | TCTTCGTCA |
| 83 | TSSTWWCWWGWSC | TCTTCTGAC |
| 84 | TSWAWSGWSCWWG | TGAAGACTG |
| 85 | TSWAWSGWWGWSC | TGAAGTGAC |
| 86 | TSWASSTSSAWWG | TGACTCATG |
| 87 | TSWASSTSWTWSC | TGACTGTAC |
| 88 | TSWCWSTWWGWWG | TGCATTGTG |
| 89 | TSWCSSGWSASWG | TGCCGAAGG |
| 90 | TSWCSWCWWTSWG | TGCGCTTGG |
| 91 | TSWCWWASWGSWG | TGCTAGGGG |
| 92 | TSWGWSAWWCWWC | TGGAATCTC |
| 93 | TSWGSWGWWASWC | TGGGGTAGC |
| 94 | TSWGWWTSWCSWC | TGGTTGCGC |
| 95 | TSWTWSCWSGWWC | TGTACAGTC |
| 96 | TSWTSSASSTWWC | TGTCACTTC |
| 97 | AWSAWSASWTSSA | AAAAAGTCA |
| 98 | AWSASWGSWCSWC | AAAGGGCGC |
| 99 | AWSAWWTSSTWWC | AAATTCTTC |
| 100 | AWSCWWGWSTSWC | AACTGATGC |
| 101 | AWSTWSTWSASWG | AATATAAGG |
| 102 | AWSTSWCWSCWWG | AATGCACTG |
| 103 | ASSAWSCWSGWWC | ACAACAGTC |
| 104 | ASSAWSCSWGWSA | ACAACGGAA |
| 105 | ASSASWTWSCWWG | ACAGTACTG |
| 106 | ASSASWTSWGSWG | ACAGTGGGG |
| 107 | ASSASWTSWTSSA | ACAGTGTCA |
| 108 | ASSGWSTSSASSA | ACGATCACA |
| 109 | ASSTSSTSSAWWG | ACTCTCATG |
| 110 | ASSTSSTSWTWSC | ACTCTGTAC |
| 111 | ASWAWSGWWGWWG | AGAAGTGTG |
| 112 | ASWASWASSAWWG | AGAGACATG |
| 113 | ASWCWSTWSCWWG | AGCATACTG |
| 114 | ASWCWSTSWGSWG | AGCATGGGG |
| 115 | ASWCSSGWWCWWC | AGCCGTCTC |
| 116 | ASWCSWCWSASWG | AGCGCAAGG |
| 117 | ASWCSWCSWGWSA | AGCGCGGAA |
| 118 | ASWCWWASSASSA | AGCTACACA |
| 119 | ASWGWSAWSGWWC | AGGAAAGTC |
| 120 | ASWGWSASSAWSC | AGGAACAAC |
| 121 | ASWGWWTSSAWWG | AGGTTCATG |
| 122 | ASWGWWTSWTWSC | AGGTTGTAC |
| 123 | ASWTSSAWSAWSA | AGTCAAAAA |
| 124 | CWSASWGWWASWC | CAAGGTAGC |
| 125 | CWSAWWTSWCSWC | CAATTGCGC |
| 126 | CWSCWSCSWCSWC | CACACGCGC |
| 127 | CWSGWWCSSASSA | CAGTCCACA |
| 128 | CWSGWWCSSTWWC | CAGTCCTTC |
| 129 | CWSTWSTWWGWWG | CATATTGTG |
| 130 | CWSTSSGWSASWG | CATCGAAGG |
| 131 | CWWCSWASSAWWG | CTCGACATG |
| 132 | CWWTSWGWSAWSA | CTTGGAAAA |
| 133 | CWWTSWGSWGSWG | CTTGGGGGG |
| 134 | GWSAWSASWGWSA | GAAAAGGAA |
| 135 | GWSASWGWSAWSA | GAAGGAAAA |
| 136 | GWSAWWTSSAWWG | GAATTCATG |
| 137 | GWSAWWTSWTWSC | GAATTGTAC |
| 138 | GWSCWSCWWTSWG | GACACTTGG |
| 139 | GWSCSWTWSCWSC | GACGTACAC |
| 140 | GWSCWWGSWGWSA | GACTGGGAA |
| 141 | GWSGWSGWWASWC | GAGAGTAGC |
| 142 | GWSTSSGWWTSWG | GATCGTTGG |
| 143 | GWSTSWCSWCSWC | GATGCGCGC |
| 144 | GSWASWASSTWWC | GGAGACTTC |
| 145 | GSWCWSTWSGWWC | GGCATAGTC |
| 146 | GSWCWSTSSAWSC | GGCATCAAC |
| 147 | GSWCSSGWWGWWG | GGCCGTGTG |
| 148 | GSWCSWCSWGSWG | GGCGCGGGG |
| 149 | GSWGWSAWSAWSA | GGGAAAAAA |
| 150 | GSWGWSASWGSWG | GGGAAGGGG |
| 151 | GSWGWSAWWGWSC | GGGAATGAC |
| 152 | GSWGWWTSSASSA | GGGTTCACA |
| 153 | GSWTWSCWWGWWG | GGTACTGTG |
| 154 | GSWTSSAWSGWWC | GGTCAAGTC |
| 155 | GSWTSWTSWTWSC | GGTGTGTAC |
| 156 | GWWCWSGWSAWSA | GTCAGAAAA |
| 157 | GWWCWSGWSCWWG | GTCAGACTG |
| 158 | GWWCSSTSSAWWG | GTCCTCATG |
| 159 | GWWCSWASSAWSC | GTCGACAAC |
| 160 | GWWCWWCWSCWSC | GTCTCACAC |
| 161 | GWWCWWCWWGWWG | GTCTCTGTG |
| 162 | GWWGWSCWSASWG | GTGACAAGG |
| 163 | GWWGWWGWWCWWC | GTGTGTCTC |
| 164 | TWSCWSCSWTSSA | TACACGTCA |
| 165 | TWSCWSCWWGWSC | TACACTGAC |
| 166 | TWSCSWTWSGWWC | TACGTAGTC |
| 167 | TWSCWWGWSCWSC | TACTGACAC |
| 168 | TWSTWSTWWCWWC | TATATTCTC |
| 169 | TWSTSSGWSCWWG | TATCGACTG |
| 170 | TWSTSSGWSTSWC | TATCGATGC |
| 171 | TWSTWWASSAWSC | TATTACAAC |
| 172 | TSSASSAWSAWSA | TCACAAAAA |
| 173 | TSSASSAWSCWWG | TCACAACTG |
| 174 | TSSASSASWTSSA | TCACAGTCA |
| 175 | TSSAWWGWSGWWC | TCATGAGTC |
| 176 | TSSAWWGSWCSWC | TCATGGCGC |
| 177 | TSSGWSTWSAWSA | TCGATAAAA |
| 178 | TSSGWSTWWGWSC | TCGATTGAC |
| 179 | TSSTSSTWSGWWC | TCTCTAGTC |
| 180 | TSSTSSTSSAWSC | TCTCTCAAC |
| 181 | TSSTWWCWSAWSA | TCTTCAAAA |
| 182 | TSSTWWCSWGSWG | TCTTCGGGG |
| 183 | TSWASWAWSGWWC | TGAGAAGTC |
| 184 | TSWCWSTSSASSA | TGCATCACA |
| 185 | TSWCWSTWWASWC | TGCATTAGC |
| 186 | TSWCSWCSWTWSC | TGCGCGTAC |
| 187 | TSWCSWCWWCWWC | TGCGCTCTC |
| 188 | TSWGWSASSAWWG | TGGAACATG |
| 189 | TSWGSWGWSCWSC | TGGGGACAC |
| 190 | TSWGWWTSSAWSC | TGGTTCAAC |
| 191 | TSWTWSCSWCSWC | TGTACGCGC |
| 192 | TSWTSSAWWGWWG | TGTCATGTG |
| 193 | AWSAWSAWSCWWG | AAAAAACTG |
| 194 | AWSASWGWSGWWC | AAAGGAGTC |
| 195 | AWSAWWTSSASSA | AAATTCACA |
| 196 | AWSCWSCWWASWC | AACACTAGC |
| 197 | AWSGWSGWWCWWC | AAGAGTCTC |
| 198 | AWSGWWCSSAWSC | AAGTCCAAC |
| 199 | AWSGWWCSWCSWC | AAGTCGCGC |
| 200 | AWSTWSTWSGWWC | AATATAGTC |
| 201 | AWSTWSTSWGWSA | AATATGGAA |
| 202 | AWSTSSGWSCWSC | AATCGACAC |
| 203 | AWSTSSGWWGWWG | AATCGTGTG |
| 204 | ASSAWSCWSASWG | ACAACAAGG |
| 205 | ASSASSASSTWWC | ACACACTTC |
| 206 | ASSGWSTSSTWWC | ACGATCTTC |
| 207 | ASSGWSTWWGWWG | ACGATTGTG |
| 208 | ASSTSWAWSASWG | ACTGAAAGG |
| 209 | ASSTSWASSAWSC | ACTGACAAC |
| 210 | ASSTSWASWGWSA | ACTGAGGAA |
| 211 | ASWAWSGWSCWSC | AGAAGACAC |
| 212 | ASWCWWASSTWWC | AGCTACTTC |
| 213 | ASWGWSASWCSWC | AGGAAGCGC |
| 214 | ASWGSWGWSTSWC | AGGGGATGC |
| 215 | ASWTSSAWSTSWC | AGTCAATGC |
| 216 | ASWTSSASWGSWG | AGTCAGGGG |
| 217 | CWSAWWTSWGWSA | CAATTGGAA |
| 218 | CWSGWSGWSAWSA | CAGAGAAAA |
| 219 | CWSGWSGWWGWSC | CAGAGTGAC |
| 220 | CWSGWWCWWASWC | CAGTCTAGC |
| 221 | CWSGWWCWWGWWG | CAGTCTGTG |
| 222 | CWSTWSTWSCWSC | CATATACAC |
| 223 | CWSTWSTWWASWC | CATATTAGC |
| 224 | CWSTSWCSSAWWG | CATGCCATG |
| 225 | CWSTSWCWWCWWC | CATGCTCTC |
| 226 | CWWASSGWWTSWG | CTACGTTGG |
| 227 | CWWASWCSSAWSC | CTAGCCAAC |
| 228 | CWWASWCSWCSWC | CTAGCGCGC |
| 229 | CWWCSSTSWGWSA | CTCCTGGAA |
| 230 | CWWCWWCWSAWSA | CTCTCAAAA |
| 231 | CWWGWSCSWTWSC | CTGACGTAC |
| 232 | CWWGWWGWSASWG | CTGTGAAGG |
| 233 | CWWTSWGWSTSWC | CTTGGATGC |
| 234 | GWSAWSAWSASWG | GAAAAAAGG |
| 235 | GWSASWGWSCWWG | GAAGGACTG |
| 236 | GWSASWGWWGWSC | GAAGGTGAC |
| 237 | GWSCWWGWSASWG | GACTGAAGG |
| 238 | GWSGWSGWSCWSC | GAGAGACAC |
| 239 | GWSGWWCWWGWSC | GAGTCTGAC |
| 240 | GWSTWSTWSAWSA | GATATAAAA |
| 241 | GWSTWSTWSTSWC | GATATATGC |
| 242 | GWSTSSGWWCWWC | GATCGTCTC |
| 243 | GWSTSWCWSASWG | GATGCAAGG |
| 244 | GWSTSWCWSGWWC | GATGCAGTC |
| 245 | GSWASSTWSTSWC | GGACTATGC |
| 246 | GSWCWSTWSASWG | GGCATAAGG |
| 247 | GSWGWSASWTSSA | GGGAAGTCA |
| 248 | GSWGSWGWSASWG | GGGGGAAGG |
| 249 | GSWTSSAWSASWG | GGTCAAAGG |
| 250 | GSWTSSASSAWSC | GGTCACAAC |
| 251 | GSWTSSASWGWSA | GGTCAGGAA |
| 252 | GSWTSWTSSAWWG | GGTGTCATG |
| 253 | GWWASSGWSGWWC | GTACGAGTC |
| 254 | GWWASWCWWCWWC | GTAGCTCTC |
| 255 | GWWCWSGWWGWSC | GTCAGTGAC |
| 256 | GWWGWSCWSGWWC | GTGACAGTC |
| 257 | GWWGWSCSWGWSA | GTGACGGAA |
| 258 | GWWGSWTWSCWWG | GTGGTACTG |
| 259 | GWWGSWTSWGSWG | GTGGTGGGG |
| 260 | GWWTSWGWWGWWG | GTTGGTGTG |
| 261 | TWSAWSAWSCWSC | TAAAAACAC |
| 262 | TWSAWSASSTWWC | TAAAACTTC |
| 263 | TWSASWGWWTSWG | TAAGGTTGG |
| 264 | TWSAWWTSWGSWG | TAATTGGGG |
| 265 | TWSCWSCWSAWSA | TACACAAAA |
| 266 | TWSCWSCWSCWWG | TACACACTG |
| 267 | TWSCWSCSWGSWG | TACACGGGG |
| 268 | TWSCSWTWSASWG | TACGTAAGG |
| 269 | TWSCSWTSWGWSA | TACGTGGAA |
| 270 | TWSCWWGWWASWC | TACTGTAGC |
| 271 | TWSGWSGWSASWG | TAGAGAAGG |
| 272 | TWSGWSGWSGWWC | TAGAGAGTC |
| 273 | TWSGWWCSWTWSC | TAGTCGTAC |
| 274 | TWSGWWCWWCWWC | TAGTCTCTC |
| 275 | TWSTSWCSSASSA | TATGCCACA |
| 276 | TWSTSWCWWASWC | TATGCTAGC |
| 277 | TSSASSAWSTSWC | TCACAATGC |
| 278 | TSSASSASWGSWG | TCACAGGGG |
| 279 | TSSASWTSSASSA | TCAGTCACA |
| 280 | TSSTWSGWWASWC | TCTAGTAGC |
| 281 | TSWASWAWSASWG | TGAGAAAGG |
| 282 | TSWCWSTWSCWSC | TGCATACAC |
| 283 | TSWGSWGWWGWWG | TGGGGTGTG |
| 284 | TSWGWWTSWGWSA | TGGTTGGAA |
| 285 | TSWTSSAWSCWSC | TGTCAACAC |
| 286 | TSWTSWTWSAWSA | TGTGTAAAA |
| 287 | TSWTSWTWSCWWG | TGTGTACTG |
| 288 | TSWTSWTSWGSWG | TGTGTGGGG |
| 289 | AWSAWSAWWGWSC | AAAAATGAC |
| 290 | AWSCWSCWWGWWG | AACACTGTG |
| 291 | AWSGWWCWSASWG | AAGTCAAGG |
| 292 | AWSGWWCSWGWSA | AAGTCGGAA |
| 293 | AWSTSSGWWASWC | AATCGTAGC |
| 294 | AWSTSWCWSAWSA | AATGCAAAA |
| 295 | AWSTSWCSWGSWG | AATGCGGGG |
| 296 | AWSTSWCWWGWSC | AATGCTGAC |
| 297 | ASSASWTWSAWSA | ACAGTAAAA |
| 298 | ASSASWTWSTSWC | ACAGTATGC |
| 299 | ASSGWSTWSCWSC | ACGATACAC |
| 300 | ASSGWWASWGSWG | ACGTAGGGG |
| 301 | ASSTWSGWSCWWG | ACTAGACTG |
| 302 | ASSTWSGWSTSWC | ACTAGATGC |
| 303 | ASSTWSGWWGWSC | ACTAGTGAC |
| 304 | ASSTWWCSSASSA | ACTTCCACA |
| 305 | ASSTWWCWWGWWG | ACTTCTGTG |
| 306 | ASWAWSGWWASWC | AGAAGTAGC |
| 307 | ASWASSTSWCSWC | AGACTGCGC |
| 308 | ASWCWSTSWTSSA | AGCATGTCA |
| 309 | ASWGSWGWSCWWG | AGGGGACTG |
| 310 | ASWGSWGSWTSSA | AGGGGGTCA |
| 311 | ASWGSWGWWGWSC | AGGGGTGAC |
| 312 | ASWTWSCWWCWWC | AGTACTCTC |
| 313 | ASWTSWTSSASSA | AGTGTCACA |
| 314 | ASWTSWTSSTWWC | AGTGTCTTC |
| 315 | CWSAWSAWWTSWG | CAAAATTGG |
| 316 | CWSASWGWWGWWG | CAAGGTGTG |
| 317 | CWSCWWGWWCWWC | CACTGTCTC |
| 318 | CWSCWWGWWTSWG | CACTGTTGG |
| 319 | CWSGWSGWSCWWG | CAGAGACTG |
| 320 | CWSTWSTSSASSA | CATATCACA |
| 321 | CWSTSWCSWTWSC | CATGCGTAC |
| 322 | CWSTSWCWWTSWG | CATGCTTGG |
| 323 | CWSTWWASWGSWG | CATTAGGGG |
| 324 | CWSTWWASWTSSA | CATTAGTCA |
| 325 | CWWASWCWSASWG | CTAGCAAGG |
| 326 | CWWCWSGWSCWSC | CTCAGACAC |
| 327 | CWWCSSTWSGWWC | CTCCTAGTC |
| 328 | CWWCSSTSSAWSC | CTCCTCAAC |
| 329 | CWWCSWASWTWSC | CTCGAGTAC |
| 330 | CWWGWSCWWCWWC | CTGACTCTC |
| 331 | CWWGSWTSSTWWC | CTGGTCTTC |
| 332 | CWWGWWGWSGWWC | CTGTGAGTC |
| 333 | CWWGWWGSWCSWC | CTGTGGCGC |
| 334 | GWSAWSASSAWSC | GAAAACAAC |
| 335 | GWSAWSASWCSWC | GAAAAGCGC |
| 336 | GWSCWSCSWTWSC | GACACGTAC |
| 337 | GWSCSWTSSASSA | GACGTCACA |
| 338 | GWSCWWGWSGWWC | GACTGAGTC |
| 339 | GWSGWWCWSAWSA | GAGTCAAAA |
| 340 | GWSGWWCWSCWWG | GAGTCACTG |
| 341 | GWSGWWCWSTSWC | GAGTCATGC |
| 342 | GWSTWSTWSCWWG | GATATACTG |
| 343 | GWSTWSTSWGSWG | GATATGGGG |
| 344 | GWSTWWASSTWWC | GATTACTTC |
| 345 | GSWASSTSWTSSA | GGACTGTCA |
| 346 | GSWASWAWSCWSC | GGAGAACAC |
| 347 | GSWASWASSASSA | GGAGACACA |
| 348 | GSWCSSGWWASWC | GGCCGTAGC |
| 349 | GSWCSWCWSCWWG | GGCGCACTG |
| 350 | GSWGSWGSWCSWC | GGGGGGCGC |
| 351 | GWWASSGWSASWG | GTACGAAGG |
| 352 | GWWASWCWWTSWG | GTAGCTTGG |
| 353 | GWWCWSGWSTSWC | GTCAGATGC |
| 354 | GWWCSSTSWTWSC | GTCCTGTAC |
| 355 | GWWCSSTWWCWWC | GTCCTTCTC |
| 356 | GWWCSWASWCSWC | GTCGAGCGC |
| 357 | GWWCWWCSSTWWC | GTCTCCTTC |
| 358 | GWWCWWCWWASWC | GTCTCTAGC |
| 359 | GWWGSWTWSAWSA | GTGGTAAAA |
| 360 | TWSASWGSWTWSC | TAAGGGTAC |
| 361 | TWSCWSCWSTSWC | TACACATGC |
| 362 | TWSCSWTSSAWSC | TACGTCAAC |
| 363 | TWSCSWTSWCSWC | TACGTGCGC |
| 364 | TWSGWWCWWTSWG | TAGTCTTGG |
| 365 | TWSTWSTSSAWWG | TATATCATG |
| 366 | TWSTWSTSWTWSC | TATATGTAC |
| 367 | TWSTSWCWSCWSC | TATGCACAC |
| 368 | TSSAWSCWWTSWG | TCAACTTGG |
| 369 | TSSASSAWWGWSC | TCACATGAC |
| 370 | TSSASWTWSCWSC | TCAGTACAC |
| 371 | TSSAWWGWSASWG | TCATGAAGG |
| 372 | TSSGWSTWSCWWG | TCGATACTG |
| 373 | TSSTWSGWWGWWG | TCTAGTGTG |
| 374 | TSSTSSTWSASWG | TCTCTAAGG |
| 375 | TSSTSWASWTWSC | TCTGAGTAC |
| 376 | TSWAWSGWSAWSA | TGAAGAAAA |
| 377 | TSWASSTWWCWWC | TGACTTCTC |
| 378 | TSWASWASSAWSC | TGAGACAAC |
| 379 | TSWASWASWGWSA | TGAGAGGAA |
| 380 | TSWCSWCSSAWWG | TGCGCCATG |
| 381 | TSWGWSAWWTSWG | TGGAATTGG |
| 382 | TSWTWSCWSASWG | TGTACAAGG |
| 383 | TSWTSSASSASSA | TGTCACACA |
| 384 | TSWTSWTSWTSSA | TGTGTGTCA |

As an example, the following ten members were read off of the sequencer FASTQ files

(SEQ ID NO: 4)
AACATGAACATCA
(SEQ ID NO: 5)
AACATGAACATGA
(SEQ ID NO: 6)
AACATGAACAACA
(SEQ ID NO: 7)
AACATGAAGATCA
(SEQ ID NO: 8)
ATCAACAAGAAGA
(SEQ ID NO: 9)
AAGATGAACATGA
(SEQ ID NO: 10)
AAGAAGAACATGA
(SEQ ID NO: 11)
AAGATGAACATCA
(SEQ ID NO: 12)
AAGATCAACATGA
(SEQ ID NO: 13)
AAGATGAACAAGA

All of them belong to the following family, which is identified as the first of the 384 families provided:

AWSAWSAWSAWSA (family code)
=AAAAAAAAA (family identifier)

As another example, the following ten members were read off the sequencer FASTQ files:

(SEQ ID NO: 14)
AACATGACTGGTG
(SEQ ID NO: 15)
AACATGACTGGAG
(SEQ ID NO: 16)
AACATGACTGCTG
(SEQ ID NO: 17)
AACATGACTGCAG
(SEQ ID NO: 18)
AACATGACAGGTG
(SEQ ID NO: 19)
AACATGAGAGGTG
(SEQ ID NO: 20)
AACATGAGTGGTG
(SEQ ID NO: 21)
ATCATGACTGGTG
(SEQ ID NO: 22)
ATGATGACTGGTG
(SEQ ID NO: 23)
AAGATGACTGGTG

All of them belong to the following family, which is identified as the second of the 384 families provided:

AWSAWSASWGSWG (family code)
=AAAAAGGGG (family identifier)

384 families were identified for all members that were read from the sequencer FASTQ files (only 2/384 examples were provided with 10 exemplary members each). For each family there are 256 members total.

Only one of the 3 family blocks were shown here, but the family identification is carried out for all members across all 3 of the family bocks. The combination of the 3×384 family provide 56 623 104 full length family bead barcodes in total. The 13 bp in the block sequences was chosen as a length to provide a hamming distance of 3 from each other for all the family identifier sequences and be able to obtain 384 family identifiers total. The order starting and ending with a non-wobble bp sequence aided the identification of the blocks from the FASTQ files.

For each bead type, 10,000 beads were incubated with 1 μg K562 Total RNA (Ambion) at 25° C. for 25 min. Tubes were then incubated on ice an additional 10 min. Bead were washed 3× and resuspended in 1×SSVI RT buffer. Beads were pelleted by centrifugation, the supernatant removed, and resuspended in 100 μl of an RT cocktail.

Beads were incubated at 55° C. for 16 min, washed 1× with 200 μl PBS, then washed 1× with 200 μl water and the pellet resuspended in 20 μl water. The entire 20 μl volume was transferred to a PCR tube for PCR.

Samples were then amplified for 4+7 cycles using the following protocol:

| | | Time | |
|---|---|---|---|
| Cycles | Temp | (s) | Stage |
| 1 | 95° C. | 3 min | Denature |
| 4 | 95° C. | 20 | Stage 2 |
| | 65° C. | 45 | |
| | 72° C. | 3 min | |
| 7 | 95° C. | 20 | Amp |
| | 67° C. | 25 | |
| | 72° C. | 3 min | |
| 1 | 72° C. | 10 min | Ext |
| 1 | 4° C. | hold | Hold |

Post amplification, the product was cleaned up by performing two 1.2×Ampure clean ups.

The resulting products were then processed into libraries using the NEBNext Ultra™ II FS DNA Library prep kit for Illumina using 10 ng of cDNA per reaction. The resulting libraries were sequenced on Miseq and the resulting data analyzed by the automated pipeline. Data were comparable among the bead types with the samples utilizing the dimer barcode successfully being binned to identify comparable numbers of CBCs, and were able to successfully map the detected genes to the CBCs and identify and collapse workflow duplicates which the pipeline identifies as UMIs regardless of whether they result from the dimer code or UMIs.

| | | | | Mean | % | | | Gene |
|---|---|---|---|---|---|---|---|---|
| Sample | | CBC | Total | Filtered | Genic | % Mito | Genes | Max |
| Number | Sample | Examined | Filtered | Per CBC | Reads | Reads | Mean | Output |
| S1 | Cel202009ApolyT | 3,598 | 4,189,906 | 1,165 | 45.10% | 0.64% | 401 | 961 |
| | 09_1_S1 | | | | | | | |
| S2 | Cel202009ApolyT | 4,092 | 4,587,828 | 1,121 | 42.66% | 0.63% | 370 | 1,017 |
| | 09_1_S2 | | | | | | | |
| S3 | CBR202007ApolyT | 2,979 | 2,722,305 | 914 | 43.38% | 0.61% | 304 | 1,218 |
| | CL_1_S3 | | | | | | | |
| S4 | CBR202007ApolyT | 3,139 | 3,791,624 | 1,208 | 43.32% | 0.63% | 378 | 1,258 |
| | CL_2_S4 | | | | | | | |
| S5 | CBR202011Apoly | 2,271 | 3,113,256 | 1,371 | 45.79% | 0.53% | 472 | 1,121 |
| | TCLHam_1_S5 | | | | | | | |
| S6 | CBR202011Apoly | 2,240 | 3,733,221 | 1,667 | 45.61% | 0.52% | 552 | 1,509 |
| | TCLHam_2_S6 | | | | | | | |

| | | | | | | | | | % | % |
|---|---|---|---|---|---|---|---|---|---|---|
| | | UMI | Genic | | | % | % | % | Low | Ribo- |
| Sample | UMI | Max | Reads | % | % | Intro- | Inter- | Ambig- | Map | somal |
| Number | Mean | Output | Mean | Coding | UTR | nic | genic | uous | Qual | Protein |
| S1 | 515 | 1,520 | 525 | 40.72% | 22.60% | 8.95% | 2.13% | 2.74% | 47.60% | 8.55% |
| S2 | 469 | 1,624 | 478 | 38.62% | 21.49% | 8.71% | 2.07% | 2.59% | 50.04% | 8.14% |
| S3 | 387 | 2,039 | 396 | 39.82% | 19.84% | 9.33% | 1.76% | 2.28% | 47.93% | 7.48% |
| S4 | 507 | 2,196 | 523 | 40.02% | 19.63% | 9.41% | 1.76% | 2.34% | 47.79% | 7.58% |
| S5 | 615 | 1,857 | 628 | 41.24% | 21.90% | 9.37% | 1.90% | 2.58% | 46.04% | 7.76% |
| S6 | 742 | 2,787 | 760 | 40.76% | 21.91% | 9.34% | 1.86% | 2.53% | 46.26% | 7.56% |

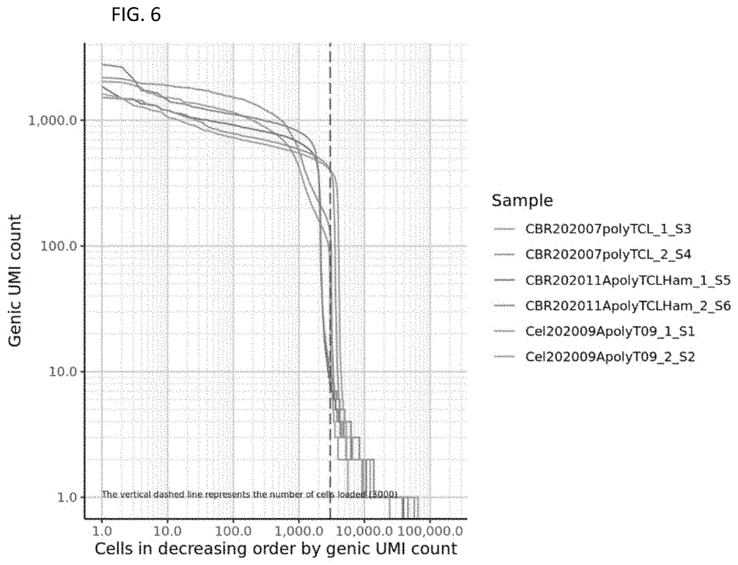

The resulting data were also plotted in knee plots (FIG. 6) to illustrate the ability to differentiate sequences originating from different beads.

Polystyrene beads with the dimer barcode described above were also used in single cell analysis on the Genesis system, and also resulted in knee plots which demonstrate the ability to map the reads to beads (and thus cells) based on the barcodes, and identify non-unique sequences comparably to a UMI.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A solid support comprising multiple copies of a plurality of at least 10 different oligonucleotide members,
wherein all oligonucleotide members encode the same family identification sequence, and
wherein the oligonucleotide members comprise one or more sequence block having at least three nucleotide positions and comprising the formula (X)ₙ(Y)ₘ or (Y)ₘ(X)ₙ, wherein X is a degenerate nucleotide, n is 2-50 (e.g., 2-20, 3-20, 4-20, 5-20), Y is constant within the oligonucleotide family, and m is 1-50 (e.g., 1-30, 1-20, 1-10, 1-5), wherein the sum of n and m is at least three, wherein degenerate nucleotides in the at least three nucleotide positions are related between oligonucleotide members by a code such that different oligonucleotide members are decoded to the same oligonucleotide family sequence.

2. The solid support of claim 1, where the solid support has between 2-1000 copies of each different oligonucleotide member.

3. The solid support of claim 1, wherein n is 2 and m is 1.

4. The solid support of claim 1, wherein the sequence block has the formula Y[(X)ₙ(Y)ₘ]_{z}, wherein z is 1, 2, 3, 4, 5, 6, 7, 8, 9, or 10.

5. The solid support of claim 4, wherein z is 4, n is 2 and m is 1.

6. The solid support of claim 1, wherein the family identification sequence of each oligonucleotide member is encoded by one or more (e.g., 1, 2 3, 4, 5, or more) sequence block comprising XₙYₘXₙ.

7. The solid support of claim 1, wherein the family identification sequence of each oligonucleotide member is encoded by one or more (e.g., 1, 2 3, 4, 5, or more) sequence block comprising YₘXₙYₘ.

8. The solid support of claim 1, wherein n is 2 or 3 or 4 and m is 1 or 2.

9. The solid support of claim 1, where the solid support has between 2-1000 (e.g., 2-50 or 2-500) copies of each different oligonucleotide member.

10. The solid support of claim 1, wherein the oligonucleotide member do not comprise a unique molecular identification (UMI) sequence separate from the family identification sequence.

11. The solid support of claim 1, wherein oligonucleotide members are composed of two or more (e.g., 2, 3, 4, 5, 6, or more) sequence blocks.

12. The solid support of claim 1, wherein the oligonucleotide members comprise a 3′ poly T sequence.

13. The solid support of claim 1, wherein oligonucleotide members are linked to the solid support.

14. The solid support of claim 1, wherein the solid support is a bead.

15. The solid support of claim 14, wherein the bead is a dissolvable bead that contains the oligonucleotide members.

16. The solid support of claim 15, wherein the oligonucleotide members are reversibly (releasably) or irreversibly linked to the bead.

17. A composition comprising a plurality of different solid supports of claim 1, wherein different beads have oligonucleotide members from different oligonucleotide families.

18. The composition of claim 17, wherein the plurality comprises at least 100, 1000, 10000 or more different solid supports.

19. The composition of claim 17, wherein oligonucleotide family sequence of different solid supports differ from all other oligonucleotide family sequences by at least two nucleotides in the family identification sequence.

20. A composition comprising a plurality of different solid supports,
wherein each solid support comprises multiple copies of a plurality of at least 10 different oligonucleotide members and all oligonucleotide members of a solid support encode the same family identification sequence; and
wherein each oligonucleotide member comprises one or more sequence block comprising two or more nucleotides, wherein the two or more nucleotides are degenerate nucleotides and related between oligonucleotide members by a code such that different oligonucleotide members are decoded to the same family identification sequence for the solid support to which the oligonucleotide members are associated;
wherein different family identification sequences of different solid supports differ from all other family identification sequences for other solid supports by at least two nucleotides.

21. A method of generating nucleotide sequences from a sample, the method comprising,
providing a plurality of partitions, wherein a partition of the plurality comprises a polynucleotide sample and the solid support of claim 17;
attaching the oligonucleotides from the bead to polynucleotides from the polynucleotide sample to form fusion polynucleotides;
nucleotide sequencing at least a portion of the fusion polynucleotide comprising the identification sequence and at least a portion of the polynucleotide from the polynucleotide sample, thereby generating sequencing reads.


SEQUENCE LISTING (23 sequences)

SEQ ID NO: 1 - DNA, 14 bp, Artificial Sequence
  Description of Artificial Sequence Synthetic oligonucleotide
gacgctgccgacga

SEQ ID NO: 2 - DNA, 15 bp, Artificial Sequence
  Description of Artificial Sequence Synthetic oligonucleotide
ccgagcccacgagac

SEQ ID NO: 3 - DNA, 118 bp, Artificial Sequence
  Description of Artificial Sequence Synthetic polynucleotide
  modified_base (26)..(38): a, c, t, g, unknown or other
  modified_base (39)..(42): This region may encompass one of the following sequences “g” or “cg” or “tcc” or “hdcg” or it may be absent
  modified_base (53)..(65): a, c, t, g, unknown or other
  modified_base (76)..(88): a, c, t, g, unknown or other
aagcagtggtatcaacgcagagtacnnnnnnnnnnnnnnnnnatgactacacnnnnnnnnnnnnntcaggacatcnnnnnnnnnnnnntttttttttttttttttttttttttttttt

SEQ ID NO: 4 - DNA, 13 bp, Artificial Sequence
  Description of Artificial Sequence Synthetic oligonucleotide
aacatgaacatca

SEQ ID NO: 5 - DNA, 13 bp, Artificial Sequence
  Description of Artificial Sequence Synthetic oligonucleotide
aacatgaacatga

SEQ ID NO: 6 - DNA, 13 bp, Artificial Sequence
  Description of Artificial Sequence Synthetic oligonucleotide
aacatgaacaaca

SEQ ID NO: 7 - DNA, 13 bp, Artificial Sequence
  Description of Artificial Sequence Synthetic oligonucleotide
aacatgaagatca

SEQ ID NO: 8 - DNA, 13 bp, Artificial Sequence
  Description of Artificial Sequence Synthetic oligonucleotide
atcaacaagaaga

SEQ ID NO: 9 - DNA, 13 bp, Artificial Sequence
  Description of Artificial Sequence Synthetic oligonucleotide
aagatgaacatga

SEQ ID NO: 10 - DNA, 13 bp, Artificial Sequence
  Description of Artificial Sequence Synthetic oligonucleotide
aagaagaacatga

SEQ ID NO: 11 - DNA, 13 bp, Artificial Sequence
  Description of Artificial Sequence Synthetic oligonucleotide
aagatgaacatca

SEQ ID NO: 12 - DNA, 13 bp, Artificial Sequence
  Description of Artificial Sequence Synthetic oligonucleotide
aagatcaacatga

SEQ ID NO: 13 - DNA, 13 bp, Artificial Sequence
  Description of Artificial Sequence Synthetic oligonucleotide
aagatgaacaaga

SEQ ID NO: 14 - DNA, 13 bp, Artificial Sequence
  Description of Artificial Sequence Synthetic oligonucleotide
aacatgactggtg

SEQ ID NO: 15 - DNA, 13 bp, Artificial Sequence
  Description of Artificial Sequence Synthetic oligonucleotide
aacatgactggag

SEQ ID NO: 16 - DNA, 13 bp, Artificial Sequence
  Description of Artificial Sequence Synthetic oligonucleotide
aacatgactgctg

SEQ ID NO: 17 - DNA, 13 bp, Artificial Sequence
  Description of Artificial Sequence Synthetic oligonucleotide
aacatgactgcag

SEQ ID NO: 18 - DNA, 13 bp, Artificial Sequence
  Description of Artificial Sequence Synthetic oligonucleotide
aacatgacaggtg

SEQ ID NO: 19 - DNA, 13 bp, Artificial Sequence
  Description of Artificial Sequence Synthetic oligonucleotide
aacatgagaggtg

SEQ ID NO: 20 - DNA, 13 bp, Artificial Sequence
  Description of Artificial Sequence Synthetic oligonucleotide
aacatgagtggtg

SEQ ID NO: 21 - DNA, 13 bp, Artificial Sequence
  Description of Artificial Sequence Synthetic oligonucleotide
atcatgactggtg

SEQ ID NO: 22 - DNA, 13 bp, Artificial Sequence
  Description of Artificial Sequence Synthetic oligonucleotide
atgatgactggtg

SEQ ID NO: 23 - DNA, 13 bp, Artificial Sequence
  Description of Artificial Sequence Synthetic oligonucleotide
aagatgactggtg